(19) United States
(12) Patent Application Publication
(10) Pub. No.: US 2008/0318842 A1
(43) Pub. Date: Dec. 25, 2008

(54) Cyclic Isodityrosine Derivatives

(76) Inventors: Shigeru Nishiyama, Kanagawa (JP); Rika Obata, Kanagawa (JP); Hiroshi Tomoda, Tokyo (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/791,088

(22) PCT Filed: Sep. 30, 2005
(86) PCT No.: PCT/JP05/18182

§ 371 Date: Jul. 29, 2008

(30) Foreign Application Priority Data
Nov. 19, 2004 (JP) 2004-336698

(51) Int. Cl. A61K 38/12; C07K 11/02; A61P 31/04; A61P 9/10

(52) U.S. Cl. 514/9; 530/321

(57) ABSTRACT

[Problems] To provide a novel cyclic isodityrosine derivative having a physiological effect.
[Means for solving problems] A compound represented by general formula (I) or a pharmacologically acceptable salt thereof has an effect of potentiating activity of imipenem, an effect of inhibiting the synthesis of a cholesteryl ester, and so forth:

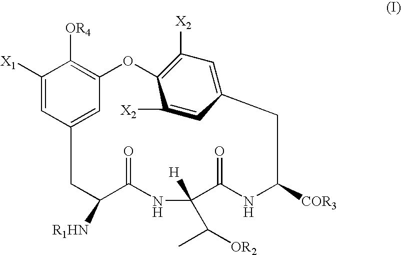

wherein R₁ represents a hydrogen atom, an amido group, a Boc group, a Cbz group, an Fmoc group, a BocNH(CH₂)₅CO— group, a BocNHCH(CH₂C₆H₅)CO— group, or a linear or branched alkyl group; R₂ represents a benzoyl group, a benzyl group, a modified benzyl group, or a linear or branched alkyl group; R₃ represents a hydrogen group, an —OR₅ group, wherein R₅ represents a linear or branched alkyl group or an aromatic ring, or an amido group; R₄ represents a hydrogen atom, or a linear or branched alkyl group; and X₁ and X₂ each represent a halogen atom, wherein X₁ and X₂ may be the same as or different from each other.

### CROSS-REFERENCE TO RELATED APPLICATION

This application claims the priority of Japanese Patent Application No. 2004-336698 filed on Nov. 19, 2004, which is hereby incorporated by reference herein in its entirety.

### TECHNICAL FIELD

The present invention relates to a cyclic isodityrosine derivative and a pharmacologically acceptable salt thereof; an imipenem activity potentiator, a cyclic isodityrosine derivative activity potentiator, an agent to inhibit the growth of bacteria, a cholesterol production suppressor, a cholesteryl ester synthesis inhibitor, and a pharmaceutical composition containing the cyclic isodityrosine derivative or a pharmacologically acceptable salt thereof as an active ingredient; and a method of potentiating activity of imipenem, a method of potentiating activity of the cyclic isodityrosine derivative, a method of inhibiting the growth of bacteria, a method of suppressing the production of cholesterol, and a method of inhibiting the production of a cholesteryl ester, using the cyclic isodityrosine derivative or a pharmacologically acceptable salt thereof.

### BACKGROUND ART

It is known that some cyclic isodityrosine derivatives have physiological effects. For example, it is known that vancomycin has anti-methicillin-resistant Staphylococcus aureus (MRSA) activity, OF-4949 has an anti-cancer effect and an effect of inhibiting aminopeptidase B, and K-13 has an effect of inhibiting the angiotensin-1-converting enzyme in hypertension (see Glycopeptide Antibiotics; Marcel Dekker, Inc.; New York, 1994, pp. 309-409, J. Antibiot. 40 (1987) 450-454, and Japanese Unexamined Patent Application Publication No. 63-203671). Examples of other cyclic isodityrosine derivatives include eurypamides A to D (see Tetrahedron Letters 44 (2003) 7949-7952 and Tetrahedron Letters 60 (2004) 5623-5634).

### SUMMARY OF THE INVENTION

It is an object of the present invention to provide a novel cyclic isodityrosine derivative having a physiological effect, a pharmacologically acceptable salt thereof, an agent and a pharmaceutical composition containing the cyclic isodityrosine derivative or a pharmacologically acceptable salt thereof as an active ingredient, and a method of using the above.

It is known that imipenem acts as an antibiotic, but does not have an effect on methicillin-resistant Staphylococcus aureus (MRSA). However, the present inventors have found that compounds represented by formulae (1) to (5) and (9) to (12) below (hereinafter referred to as “Compound (1)”, “Compound (2)”, “Compound (3)”, “Compound (4)”, “Compound (5)”, “Compound (9)”, “Compound (10)”, “Compound (11)”, and “Compound (12)”, respectively) potentiate anti-methicillin-resistant Staphylococcus aureus (MRSA) activity of imipenem.

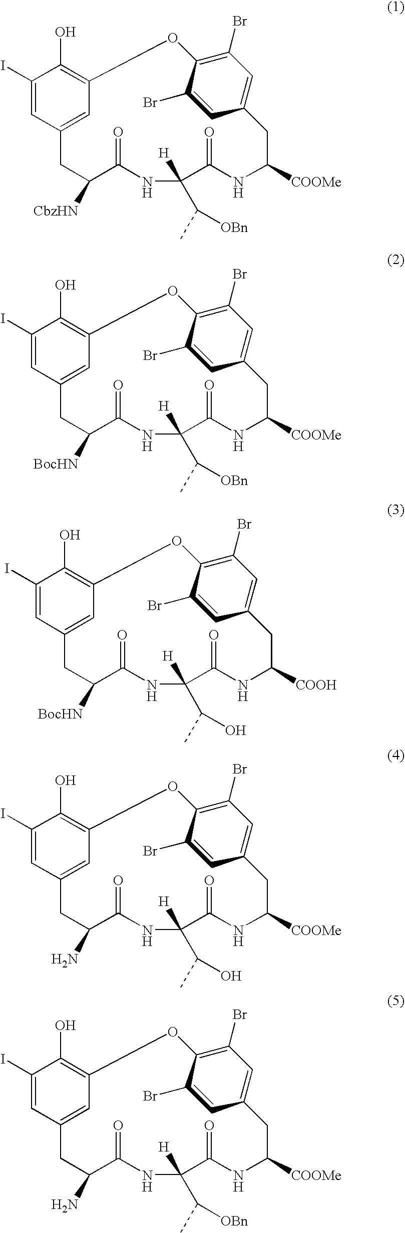

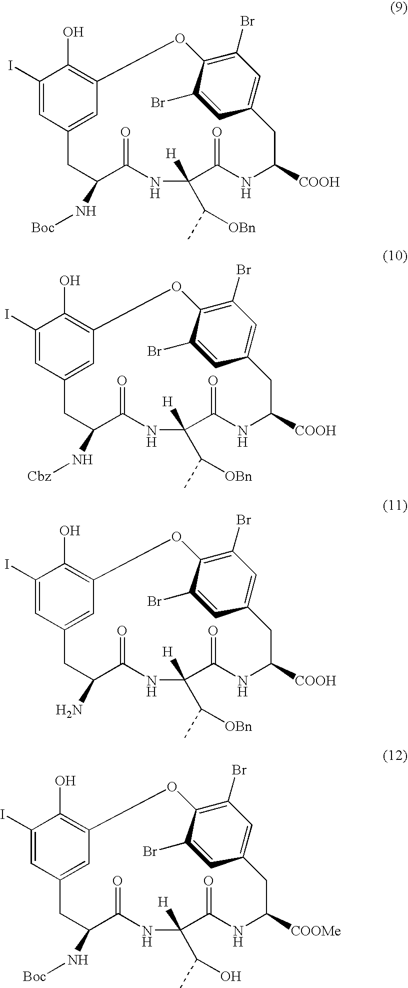

The present inventors have found that, in contrast, compounds represented by formulae (6) to (8) below (hereinafter referred to as “Compound (6)”, “Compound (7)”, and “Compound (8)”, respectively) do not potentiate anti-methicillin-resistant Staphylococcus aureus (MRSA) activity of imipenem.

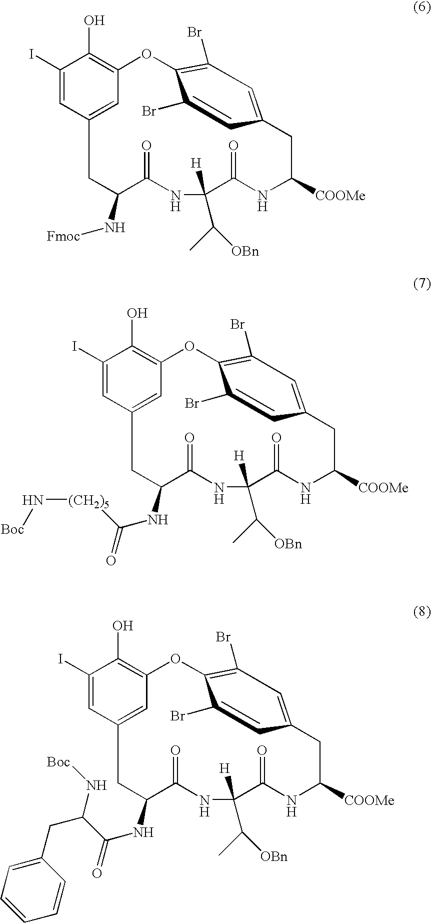

Accordingly, it is considered that compounds represented by general formula (III) below and pharmacologically acceptable salts thereof, and compounds represented by general formula (II) below and pharmacologically acceptable salts thereof have an imipenem activity-potentiating effect.

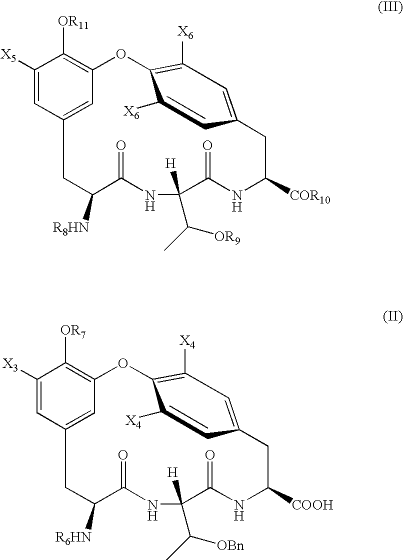

In the general formula (III), R₈ represents a hydrogen atom, an amido group, a Boc (tert-butoxycarbonyl) group, a Cbz (benzyloxycarbonyl) group, or a linear or branched alkyl group; R₉ represents a hydrogen atom, a benzoyl group, a benzyl group, a modified benzyl group, or a linear or branched alkyl group; R₁₀ represents a hydrogen atom, an —OR₁₂ group (wherein R₁₂ represents a linear or branched alkyl group or an aromatic ring), or an amido group; R₁₁ represents a hydrogen atom, or a linear or branched alkyl group; and X₅ and X₆ each represent a halogen atom, and X₅ and X₆ may be the same as or different from each other. However, when R₉ is a hydrogen atom, R₈ is a hydrogen atom or a Boc group. In the general formula (II), R₆ represents a hydrogen atom, a Boc group, or a Cbz group; R₇ represents a hydrogen atom, or a linear or branched alkyl group; and X₃ and X₄ each represent a halogen atom, and X₃ and X₄ may be the same as or different from each other.

Furthermore, the present inventors have found that Compounds (3) to (5) and Compounds (9) to (12) above do not have an effect of inhibiting the synthesis of a cholesteryl ester in macrophages, whereas Compound (1), Compound (2), Compound (6), Compound (7), and Compound (8) above have an effect of inhibiting the synthesis of a cholesteryl ester in macrophages. Accordingly, it is considered that compounds represented by general formula (IV) below wherein R₁₃ represents an amido group, a Boc group, a Cbz group, an Fmoc (9-fluorenylmethyloxycarbonyl) group, a BocNH(CH₂)₅CO— group, a BocNHCH(CH₂C₆H₅)CO— group, or a linear or branched alkyl group; R₁₄ represents a benzoyl group, a benzyl group, a modified benzyl group, or a linear or branched alkyl group; R₁₅ represents an —OR₁₇ group (wherein R₁₇ represents a linear or branched alkyl group or an aromatic ring) or an amido group; R₁₆ represents a hydrogen atom, or a linear or branched alkyl group; and X₇ and X₈ each represent a halogen atom, and X₇ and X₈ may be the same as or different from each other, and pharmacologically acceptable salts thereof can inhibit the production of a cholesteryl ester in macrophages and can suppress the production of cholesterol in macrophages.

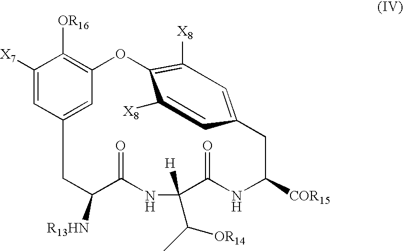

Consequently, the present inventors have found that novel cyclic isodityrosine derivatives represented by general formula (1) below or general formula (II) above, Compound (3) and Compound (4) above, and pharmacologically acceptable salts thereof have physiological effects such as an effect of potentiating activity of imipenem, an effect of inhibiting the synthesis of a cholesteryl ester, or an effect of suppressing the production of cholesterol.

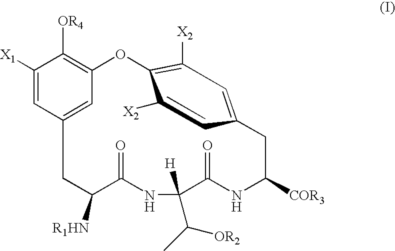

In the general formula (1), R₁ represents a hydrogen atom, an amido group, a Boc group, a Cbz group, an Fmoc group, a BocNH(CH₂)₅CO— group, a BocNHCH(CH₂C₆H₅)CO— group, or a linear or branched alkyl group; R₂ represents a benzoyl group, a benzyl group, a modified benzyl group, or a linear or branched alkyl group; R₃ represents a hydrogen group, an —OR₅ group (wherein R₅ represents a linear or branched alkyl group or an aromatic ring), or an amido group; R₄ represents a hydrogen atom, or a linear or branched alkyl group; and X₁ and X₂ each represent a halogen atom, and X₁ and X₂ may be the same as or different from each other.

As described so far, the present inventors have completed the present invention.

Namely, the present invention provides a compound represented by the general formula (1) and a pharmacologically acceptable salt thereof. In the general formula (1), R₁ represents a hydrogen atom, an amido group, a Boc group, a Cbz group, an Fmoc group, a BocNH(CH₂)₅CO— group, a BocNHCH(CH₂C₆H₅)CO— group, or a linear or branched alkyl group; R₂ represents a benzoyl group, a benzyl group, a modified benzyl group, or a linear or branched alkyl group; R₃ represents a hydrogen group, an —OR₅ group (wherein R₅ represents a linear or branched alkyl group or an aromatic ring), or an amido group; R₄ represents a hydrogen atom, or a linear or branched alkyl group; and X₁ and X₂ each represent a halogen atom, and X₁ and X₂ may be the same as or different from each other.

The present invention provides Compounds (1) to (5) above and pharmacologically acceptable salts thereof. Furthermore, the present invention provides Compounds (6) to (8) above and pharmacologically acceptable salts thereof.

The present invention provides a compound represented by the general formula (II) and a pharmacologically acceptable salt thereof. In the general formula (II), R₆ represents a hydrogen atom, a Boc group, or a Cbz group; R₇ represents a hydrogen atom, or a linear or branched alkyl group; and X₃ and X₄ each represent a halogen atom, and X₃ and X₄ may be the same as or different from each other. The present invention provides Compounds (9) to (11) above and pharmacologically acceptable salts thereof.

An imipenem activity potentiator to potentiate activity of imipenem according to the present invention contains a compound represented by the general formula (III) or a pharmacologically acceptable salt thereof as an active ingredient. In the general formula (III), R₈ represents a hydrogen atom, an amido group, a Boc group, a Cbz group, or a linear or branched alkyl group; R₉ represents a hydrogen atom, a benzoyl group, a benzyl group, a modified benzyl group, or a linear or branched alkyl group; R₁₀ represents a hydrogen atom, an —OR₁₂ group (wherein R₁₂ represents a linear or branched alkyl group or an aromatic ring), or an amido group; R₁₁ represents a hydrogen atom, or a linear or branched alkyl group; and X₅ and X₆ each represent a halogen atom, and X₅ and X₆ may be the same as or different from each other. However, when R₉ is a hydrogen atom, R₈ is a hydrogen atom or a Boc group.

An imipenem activity potentiator according to the present invention contains any one of Compounds (1) to (5) and (12) above or a pharmacologically acceptable salt thereof as an active ingredient.

Furthermore, an imipenem activity potentiator according to the present invention contains a compound represented by the general formula (II) or a pharmacologically acceptable salt thereof as an active ingredient. In the general formula (II), R₆ represents a hydrogen atom, a Boc group, or a Cbz group; R₇ represents a hydrogen atom, or a linear or branched alkyl group; and X₃ and X₄ each represent a halogen atom, and X₃ and X₄ may be the same as or different from each other.

An imipenem activity potentiator according to the present invention contains any one of Compounds (9) to (11) above or a pharmacologically acceptable salt thereof as an active ingredient.

The activity of imipenem may be, for example, antibacterial activity against bacteria. Examples of the bacteria include MRSA, Gram-negative bacteria, and Gram-positive bacteria.

A cholesterol production suppressor according to the present invention contains a compound represented by the general formula (IV) or a pharmacologically acceptable salt thereof as an active ingredient. In the general formula (IV), R₁₃ represents an amido group, a Boc group, a Cbz group, an Fmoc group, a BocNH(CH₂)₅CO— group, a BocNHCH(CH₂C₆H₅)CO— group, or a linear or branched alkyl group; R₁₄ represents a benzoyl group, a benzyl group, a modified benzyl group, or a linear or branched alkyl group; R₁₅ represents an —OR₁₇ group (wherein R₁₇ represents a linear or branched alkyl group or an aromatic ring) or an amido group; R₁₆ represents a hydrogen atom, or a linear or branched alkyl group; and X₇ and X₈ each represent a halogen atom, and X₇ and X₈ may be the same as or different from each other. Also, a cholesterol production suppressor according to the present invention contains Compound (1) or Compound (2) above, or a pharmacologically acceptable salt thereof as an active ingredient. Furthermore, a cholesterol production suppressor of the present invention contains any one of Compounds (6) to (8) above or a pharmacologically acceptable salt thereof as an active ingredient.

A cholesteryl ester production suppressor according to the present invention contains a compound represented by the general formula (IV) or a pharmacologically acceptable salt thereof as an active ingredient. In the general formula (IV), R₁₃ represents an amido group, a Boc group, a Cbz group, an Fmoc group, a BocNH(CH₂)₅CO— group, a BocNHCH(CH₂C₆H₅)CO— group, or a linear or branched alkyl group; R₁₄ represents a benzoyl group, a benzyl group, a modified benzyl group, or a linear or branched alkyl group; R₁₅ represents an —OR₁₇ group (wherein R₁₇ represents a linear or branched alkyl group or an aromatic ring) or an amido group; R₁₆ represents a hydrogen atom, or a linear or branched alkyl group; and X₇ and X₈ each represent a halogen atom, and X₇ and X₈ may be the same as or different from each other. A cholesteryl ester production inhibitor according to the present invention contains Compound (1) or Compound (2) above, or a pharmacologically acceptable salt thereof as an active ingredient. Furthermore, a cholesteryl ester production inhibitor of the present invention contains any one of Compounds (6) to (8) above or a pharmacologically acceptable salt thereof as an active ingredient.

An agent to inhibit the growth of bacteria according to the present invention contains a compound represented by the general formula (III) or a pharmacologically acceptable salt thereof, and imipenem as active ingredients. In the general formula (III), R₈ represents a hydrogen atom, an amido group, a Boc group, a Cbz group, or a linear or branched alkyl group; R₉ represents a hydrogen atom, a benzoyl group, a benzyl group, a modified benzyl group, or a linear or branched alkyl group; R₁₀ represents a hydrogen atom, an —OR₁₂ group (wherein R₁₂ represents a linear or branched alkyl group or an aromatic ring), or an amido group; R₁₁ represents a hydrogen atom, or a linear or branched alkyl group; and X₅ and X₆ each represent a halogen atom, and X₅ and X₆ may be the same as or different from each other. However, when R₉ is a hydrogen atom, R₈ is a hydrogen atom or a Boc group.

An agent to inhibit the growth of bacteria according to the present invention contains any one of Compounds (1) to (5) and (12) above or a pharmacologically acceptable salt thereof, and imipenem as active ingredients.

Furthermore, an agent to inhibit the growth of bacteria according to the present invention contains a compound represented by the general formula (II) or a pharmacologically acceptable salt thereof, and imipenem as active ingredients. In general formula (II), R₆ represents a hydrogen atom, a Boc group, or a Cbz group; R₇ represents a hydrogen atom, or a linear or branched alkyl group; and X₃ and X₄ each represent a halogen atom, and X₃ and X₄ may be the same as or different from each other.

An agent to inhibit the growth of bacteria according to the present invention contains any one of Compounds (9) to (11) above or a pharmacologically acceptable salt thereof, and imipenem as active ingredients.

Examples of the bacteria include MRSA, Gram-negative bacteria, and Gram-positive bacteria.

A pharmaceutical composition to treat a disease caused by the infection or the growth of bacteria according to the present invention contains a compound represented by the general formula (III) or a pharmacologically acceptable salt thereof, and imipenem as active ingredients. In the general formula (III), R₈ represents a hydrogen atom, an amido group, a Boc group, a Cbz group, or a linear or branched alkyl group; R₉ represents a hydrogen atom, a benzoyl group, a benzyl group, a modified benzyl group, or a linear or branched alkyl group; R₁₀ represents a hydrogen atom, an —OR₁₂ group (wherein R₁₂ represents a linear or branched alkyl group or an aromatic ring), or an amido group; R₁₁ represents a hydrogen atom, or a linear or branched alkyl group; and X₅ and X₆ each represent a halogen atom, and X₅ and X₆ may be the same as or different from each other. However, when R₉ is a hydrogen atom, R₈ is a hydrogen atom or a Boc group.

A pharmaceutical composition to treat a disease caused by the infection or the growth of bacteria according to the present invention contains any one of Compounds (1) to (5) and (12) above or a pharmacologically acceptable salt thereof, and imipenem as active ingredients.

Furthermore, a pharmaceutical composition to treat a disease caused by the infection or the growth of bacteria according to the present invention contains a compound represented by the general formula (II) or a pharmacologically acceptable salt thereof, and imipenem as active ingredients. In the general formula (II), R₆ represents a hydrogen atom, a Boc group, or a Cbz group; R₇ represents a hydrogen atom, or a linear or branched alkyl group; and X₃ and X₄ each represent a halogen atom, and X₃ and X₄ may be the same as or different from each other.

A pharmaceutical composition to treat a disease caused by the infection or the growth of bacteria according to the present invention contains any one of Compounds (9) to (11) above or a pharmacologically acceptable salt thereof, and imipenem as active ingredients.

Examples of the bacteria include MRSA, Gram-negative bacteria, and Gram-positive bacteria. Examples of the diseases caused by the infection or the growth of MRSA (hereinafter may be referred to as “MRSA infections”) include encephalitis, pneumonia, septicemia, peritonitis, enteritis, osteomyelitis, cholangitis, purulent skin lesions, decubitus ulcer infections, and food poisoning and toxic shock syndrome that are caused by a toxin produced by MRSA (such as enterotoxin or toxic shock syndrome toxin-1 (TSST-1)). Examples of the diseases caused by the infection or the growth of Gram-positive bacteria or Gram-negative bacteria (hereinafter may be referred to as “Gram-positive bacterial infections or Gram-negative bacterial infections”) include toxicosis, periodontitis, inflammatory diseases, vasculitis, IV-type allergic disease, staphylococcal scalded skin syndrome, Ritter's disease, impetigo bullosa of newborns, tumors, pneumonia, arthritis, meningitis, various purulent diseases, enteritis, meningitis, bacteremia, ocular infections, food poisoning, respiratory tract infections, otitis media, sinusitis, pharyngitis, scarlet fever, acute glomerulonephritis, rheumatic fever, impetigo, fulminant infections, tooth decay, urinary tract infections, wound infections, biliary tract infections, and aggravation of atopic diseases (such as atopic dermatitis) due to bacterial infection.

A pharmaceutical composition to treat a disease caused by an increase in cholesterol according to the present invention contains a compound represented by the general formula (IV) or a pharmacologically acceptable salt thereof as an active ingredient. In the general formula (IV), R₁₃ represents an amido group, a Boc group, a Cbz group, an Fmoc group, a BocNH(CH₂)₅CO— group, a BocNHCH(CH₂C₆H₅)CO— group, or a linear or branched alkyl group; R₁₄ represents a benzoyl group, a benzyl group, a modified benzyl group, or a linear or branched alkyl group; R₁₅ represents an —OR₁₇ group (wherein R₁₇ represents a linear or branched alkyl group or an aromatic ring) or an amido group; R₁₆ represents a hydrogen atom, or a linear or branched alkyl group; and X₇ and X₈ each represent a halogen atom, and X₇ and X₈ may be the same as or different from each other.

A pharmaceutical composition to treat a disease caused by an increase in cholesterol according to the present invention contains Compound (1) or Compound (2) above, or a pharmacologically acceptable salt thereof as an active ingredient. Furthermore, a pharmaceutical composition to treat a disease caused by an increase in cholesterol according to the present invention contains any one of Compounds (6) to (8) above or a pharmacologically acceptable salt thereof as an active ingredient.

Examples of the diseases caused by an increase in cholesterol include arteriosclerosis, hyperlipidemia, and coronary artery diseases.

A pharmaceutical composition to treat a disease caused by the storage of cholesteryl ester in cells according to the present invention contains a compound represented by the general formula (IV) or a pharmacologically acceptable salt thereof as an active ingredient. In the general formula (IV), R₁₃ represents an amido group, a Boc group, a Cbz group, an Fmoc group, a BocNH(CH₂)₅CO— group, a BocNHCH(CH₂C₆H₅)CO— group, or a linear or branched alkyl group; R₁₄ represents a benzoyl group, a benzyl group, a modified benzyl group, or a linear or branched alkyl group; R₁₅ represents an —OR₁₇ group (wherein R₁₇ represents a linear or branched alkyl group or an aromatic ring) or an amido group; R₁₆ represents a hydrogen atom, or a linear or branched alkyl group; and X₇ and X₈ each represent a halogen atom, and X₇ and X₈ may be the same as or different from each other.

A pharmaceutical composition to treat a disease caused by the storage of cholesteryl ester in cells according to the present invention contains Compound (1) or Compound (2) above, or a pharmacologically acceptable salt thereof as an active ingredient. Furthermore, a pharmaceutical composition to treat a disease caused by the storage of cholesteryl ester in cells according to the present invention contains any one of Compounds (6) to (8) above or a pharmacologically acceptable salt thereof as an active ingredient.

Examples of the diseases caused by the storage of cholesteryl ester in cells include Wolman disease and cholesteryl ester storage diseases.

A cyclic isodityrosine derivative activity potentiator to potentiate activity of a compound represented by the general formula (III) or a pharmacologically acceptable salt thereof according to the present invention contains imipenem as an active ingredient. In the general formula (III), R₈ represents a hydrogen atom, an amido group, a Boc group, a Cbz group, or a linear or branched alkyl group; R₉ represents a hydrogen atom, a benzoyl group, a benzyl group, a modified benzyl group, or a linear or branched alkyl group; R₁₀ represents a hydrogen atom, an —OR₁₂ group (wherein R₁₂ represents a linear or branched alkyl group or an aromatic ring), or an amido group; R₁₁ represents a hydrogen atom, or a linear or branched alkyl group; and X₅ and X₆ each represent a halogen atom, and X₅ and X₆ may be the same as or different from each other. However, when R₉ is a hydrogen atom, R₈ is a hydrogen atom or a Boc group.

A cyclic isodityrosine derivative activity potentiator to potentiate activity of any one of Compounds (1) to (5) and (12) above or a pharmacologically acceptable salt thereof according to the present invention contains imipenem as an active ingredient.

Furthermore, a cyclic isodityrosine derivative activity potentiator to potentiate activity of compounds represented by the general formula (II) or a pharmacologically acceptable salt thereof according to the present invention contains imipenem as an active ingredient. In the general formula (II), R₆ represents a hydrogen atom, a Boc group, or a Cbz group; R₇ represents a hydrogen atom, or a linear or branched alkyl group; and X₃ and X₄ each represent a halogen atom, and X₃ and X₄ may be the same as or different from each other.

A cyclic isodityrosine derivative activity potentiator to potentiate activity of any one of Compounds (9) to (11) above or a pharmacologically acceptable salt thereof according to the present invention contains imipenem as an active ingredient.

The activity may be, for example, antibacterial activity against bacteria. Examples of the bacteria include MRSA, Gram-negative bacteria, and Gram-positive bacteria.

A method of potentiating activity of imipenem according to the present invention includes allowing a compound represented by the general formula (III) or a pharmacologically acceptable salt thereof to act in combination with imipenem. In the general formula (III), R₈ represents a hydrogen atom, an amido group, a Boc group, a Cbz group, or a linear or branched alkyl group; R₉ represents a hydrogen atom, a benzoyl group, a benzyl group, a modified benzyl group, or a linear or branched alkyl group; R₁₀ represents a hydrogen atom, an —OR₁₂ group (wherein R₁₂ represents a linear or branched alkyl group or an aromatic ring), or an amido group; R₁₁ represents a hydrogen atom, or a linear or branched alkyl group; and X₅ and X₆ each represent a halogen atom, and X₅ and X₆ may be the same as or different from each other. However, when R₉ is a hydrogen atom, R₈ is a hydrogen atom or a Boc group.

A method of potentiating activity of imipenem according to the present invention includes allowing any one of Compounds (1) to (5) and (12) above or a pharmacologically acceptable salt thereof to act in combination with imipenem.

Furthermore, a method of potentiating activity of imipenem according to the present invention includes allowing a compound represented by the general formula (II) or a pharmacologically acceptable salt thereof to act in combination with imipenem. In the general formula (II), R₆ represents a hydrogen atom, a Boc group, or a Cbz group; R₇ represents a hydrogen atom, or a linear or branched alkyl group; and X₃ and X₄ each represent a halogen atom, and X₃ and X₄ may be the same as or different from each other.

A method of potentiating activity of imipenem according to the present invention includes allowing any one of Compounds (9) to (11) above or a pharmacologically acceptable salt thereof to act in combination with imipenem.

The activity may be, for example, antibacterial activity against bacteria. Examples of the bacteria include MRSA, Gram-negative bacteria, and Gram-positive bacteria.

A cyclic isodityrosine derivative activity potentiating method to potentiate activity of a compound represented by the general formula (III) or a pharmacologically acceptable salt thereof according to the present invention includes allowing imipenem to act in combination with a compound represented by the general formula (III) or a pharmacologically acceptable salt thereof. In the general formula (III), R₈ represents a hydrogen atom, an amido group, a Boc group, a Cbz group, or a linear or branched alkyl group; R₉ represents a hydrogen atom, a benzoyl group, a benzyl group, a modified benzyl group, or a linear or branched alkyl group; R₁₀ represents a hydrogen atom, an —OR₁₂ group (wherein R₁₂ represents a linear or branched alkyl group or an aromatic ring), or an amido group; R₁₁ represents a hydrogen atom, or a linear or branched alkyl group; and X₅ and X₆ each represent a halogen atom, and X₅ and X₆ may be the same as or different from each other. However, when R₉ is a hydrogen atom, R₈ is a hydrogen atom or a Boc group.

A cyclic isodityrosine derivative activity potentiating method to potentiate activity of any one of Compounds (1) to (5) and (12) above or a pharmacologically acceptable salt thereof according to the present invention includes allowing imipenem to act in combination with any one of Compounds (1) to (5) and (12) above or a pharmacologically acceptable salt thereof.

Furthermore, a cyclic isodityrosine derivative activity potentiating method to potentiate activity of a compound represented by the general formula (II) or a pharmacologically acceptable salt thereof according to the present invention includes allowing imipenem to act in combination with a compound represented by the general formula (II) or a pharmacologically acceptable salt thereof. In the general formula (II), R₆ represents a hydrogen atom, a Boc group; or a Cbz group; R₇ represents a hydrogen atom, or a linear or branched alkyl group; and X₃ and X₄ each represent a halogen atom, and X₃ and X₄ may be the same as or different from each other.

A cyclic isodityrosine derivative activity potentiating method to potentiate activity of any one of Compounds (9) to (11) above or a pharmacologically acceptable salt thereof according to the present invention includes allowing imipenem to act in combination with any one of Compounds (9) to (11) or a pharmacologically acceptable salt thereof.

The activity may be, for example, antibacterial activity against bacteria. Examples of the bacteria include MRSA, Gram-negative bacteria, and Gram-positive bacteria.

A method of inhibiting the growth of bacteria according to the present invention includes allowing a compound represented by the general formula (III) or a pharmacologically acceptable salt thereof and imipenem to act in combination with each other. In the general formula (III), R₈ represents a hydrogen atom, an amido group, a Boc group, a Cbz group, or a linear or branched alkyl group; R₉ represents a hydrogen atom, a benzoyl group, a benzyl group, a modified benzyl group, or a linear or branched alkyl group; R₁₀ represents a hydrogen atom, an —OR₁₂ group (wherein R₁₂ represents a linear or branched alkyl group or an aromatic ring), or an amido group; R₁₁ represents a hydrogen atom, or a linear or branched alkyl group; and X₅ and X₆ each represent a halogen atom, and X₅ and X₆ may be the same as or different from each other. However, when R₉ is a hydrogen atom, R₈ is a hydrogen atom or a Boc group.

A method of inhibiting the growth of bacteria according to the present invention includes allowing any one of Compounds (1) to (5) and (12) above or a pharmacologically acceptable salt thereof and imipenem to act in combination with each other.

Furthermore, a method of inhibiting the growth of bacteria according to the present invention includes allowing a compound represented by the general formula (II) or a pharmacologically acceptable salt thereof and imipenem to act in combination with each other. In the general formula (II), R₆ represents a hydrogen atom, a Boc group, or a Cbz group; R₇ represents a hydrogen atom, or a linear or branched alkyl group; and X₃ and X₄ each represent a halogen atom, and X₃ and X₄ may be the same as or different from each other.

A method of inhibiting the growth of bacteria according to the present invention includes allowing any one of Compounds (9) to (11) above or a pharmacologically acceptable salt thereof and imipenem to act in combination with each other.

Examples of the target bacteria include MRSA, Gram-negative bacteria, and Gram-positive bacteria.

A method of suppressing the production of cholesterol according to the present invention includes administering a compound represented by the general formula (IV) or a pharmacologically acceptable salt thereof to cholesterol-producing cells such as macrophages. In general formula (IV), R₁₃ represents an amido group, a Boc group, a Cbz group, an Fmoc group, a BocNH(CH₂)₅CO— group, a BocNHCH(CH₂C₆H₅)CO— group, or a linear or branched alkyl group; R₁₄ represents a benzoyl group, a benzyl group, a modified benzyl group, or a linear or branched alkyl group; R₁₅ represents an —OR₁₇ group (wherein R₁₇ represents a linear or branched alkyl group or an aromatic ring) or an amido group; R₁₆ represents a hydrogen atom, or a linear or branched alkyl group; and X₇ and X₈ each represent a halogen atom, and X₇ and X₈ may be the same as or different from each other.

A method of suppressing the production of cholesterol according to the present invention includes administering Compound (1) or Compound (2) above, or a pharmacologically acceptable salt thereof to cholesterol-producing cells such as macrophages. Furthermore, a method of suppressing the production of cholesterol according to the present invention includes administering any one of Compounds (6) to (8) above or a pharmacologically acceptable salt thereof to cholesterol-producing cells such as macrophages.

A method of inhibiting the synthesis of a cholesteryl ester according to the present invention includes administering a compound represented by the general formula (IV) or a pharmacologically acceptable salt thereof to cholesteryl ester-producing (storing) cells such as macrophages. In the general formula (IV), R₁₃ represents an amido group, a Boc group, a Cbz group, an Fmoc group, a BocNH(CH₂)₅CO— group, a BOCNHCH(CH₂C₆H₅)CO— group, or a linear or branched alkyl group; R₁₄ represents a benzoyl group, a benzyl group, a modified benzyl group, or a linear or branched alkyl group; R₁₅ represents an —OR₁₇ group (wherein R₁₇ represents a linear or branched alkyl group or an aromatic ring) or an amido group; R₁₆ represents a hydrogen atom, or a linear or branched alkyl group; and X₇ and X₈ each represent a halogen atom, and X₇ and X₈ may be the same as or different from each other.

A method of inhibiting the synthesis of a cholesteryl ester according to the present invention includes administering Compound (1) or Compound (2) above, or a pharmacologically acceptable salt thereof to cholesteryl ester-producing (storing) cells such as macrophages. Furthermore, a method of inhibiting the production of a cholesteryl ester according to the present invention includes administering any one of Compounds (6) to (8) above or a pharmacologically acceptable salt thereof to cholesteryl ester-producing (storing) cells such as macrophages.

A method of treating a disease caused by the infection or the growth of bacteria according to the present invention includes administering a compound represented by the general formula (III) or a pharmacologically acceptable salt thereof and imipenem to a human or a vertebrate other than a human (such as a rat or a mouse) so as to act in combination with each other. In the general formula (III), R₈ represents a hydrogen atom, an amido group, a Boc group, a Cbz group, or a linear or branched alkyl group; R₉ represents a hydrogen atom, a benzoyl group, a benzyl group, a modified benzyl group, or a linear or branched alkyl group; R₁₀ represents a hydrogen atom, an —OR₁₂ group (wherein R₁₂ represents a linear or branched alkyl group or an aromatic ring), or an amido group; R₁₁ represents a hydrogen atom, or a linear or branched alkyl group; and X₅ and X₆ each represent a halogen atom, and X₅ and X₆ may be the same as or different from each other. However, when R₉ is a hydrogen atom, R₈ is a hydrogen atom or a Boc group.

A method of treating a disease caused by the infection or the growth of bacteria according to the present invention includes administering any one of Compounds (1) to (5) and (12) above or a pharmacologically acceptable salt thereof and imipenem to a human or a vertebrate other than a human (such as a rat or a mouse) so as to act in combination with each other.

A method of treating a disease caused by the infection or the growth of bacteria according to the present invention includes administering a compound represented by the general formula (II) or a pharmacologically acceptable salt thereof and imipenem to a human or a vertebrate other than a human (such as a rat or a mouse) so as to act in combination with each other. In the general formula (II), R₆ represents a hydrogen atom, a Boc group, or a Cbz group; R₇ represents a hydrogen atom, or a linear or branched alkyl group; and X₃ and X₄ each represent a halogen atom, and X₃ and X₄ may be the same as or different from each other.

A method of treating a disease caused by the infection or the growth of bacteria according to the present invention includes administering any one of Compounds (9) to (11) above or a pharmacologically acceptable salt thereof and imipenem to a human or a vertebrate other than a human (such as a rat or a mouse) so as to act in combination with each other.

Examples of the bacteria include MRSA, Gram-negative bacteria, and Gram-positive bacteria. Examples of the diseases caused by the infection or the growth of MRSA include encephalitis, pneumonia, septicemia, peritonitis, enteritis, osteomyelitis, cholangitis, purulent skin lesions, decubitus ulcer infections, and food poisoning and toxic shock syndrome that are caused by a toxin produced by MRSA (such as enterotoxin or TSST-1). Examples of the diseases caused by the infection or the growth of Gram-positive bacteria or Gram-negative bacteria include toxicosis, periodontitis, inflammatory diseases, vasculitis, IV-type allergic disease, staphylococcal scalded skin syndrome, Ritter's disease, impetigo bullosa of newborns, tumors, pneumonia, arthritis, meningitis, various purulent diseases, enteritis, meningitis, bacteremia, ocular infections, food poisoning, respiratory tract infections, otitis media, sinusitis, pharyngitis, scarlet fever, acute glomerulonephritis, rheumatic fever, impetigo, fulminant infections, tooth decay, urinary tract infections, wound infections, biliary tract infections, and aggravation of atopic diseases (such as atopic dermatitis) due to bacterial infection.

A method of treating a disease caused by an increase in cholesterol according to the present invention includes administering a compound represented by the general formula (IV) or a pharmacologically acceptable salt thereof to a human or a vertebrate other than a human (such as a rat or a mouse). In general formula (IV), R₁₃ represents an amido group, a Boc group, a Cbz group, an Fmoc group, a BocNH(CH₂)₅CO— group, a BocNHCH(CH₂C₆H₅)CO— group, or a linear or branched alkyl group; R₁₄ represents a benzoyl group, a benzyl group, a modified benzyl group, or a linear or branched alkyl group; R₁₅ represents an —OR₁₇ group (wherein R₁₇ represents a linear or branched alkyl group or an aromatic ring) or an amido group; R₁₆ represents a hydrogen atom, or a linear or branched alkyl group; and X₇ and X₈ each represent a halogen atom, and X₇ and X₈ may be the same as or different from each other.

A method of treating a disease caused by an increase in cholesterol according to the present invention includes administering Compound (1) or Compound (2) above or a pharmacologically acceptable salt thereof to a human or a vertebrate other than a human (such as a rat or a mouse). Furthermore, a method of treating a disease caused by an increase in cholesterol according to the present invention includes administering any one of Compounds (6) to (8) above or a pharmacologically acceptable salt thereof to a human or a vertebrate other than a human (such as a rat or a mouse).

Examples of the diseases caused by an increase in cholesterol include arteriosclerosis, hyperlipidemia, and coronary artery diseases.

A method of treating a disease caused by the storage of cholesteryl ester in cells according to the present invention includes administering a compound represented by the general formula (IV) or a pharmacologically acceptable salt thereof to a human or a vertebrate other than a human (such as a rat or a mouse). In the general formula (IV), R₁₃ represents an amido group, a Boc group, a Cbz group, an Fmoc group, a BocNH(CH₂)₅CO— group, a BocNHCH(CH₂C₆H₅)CO— group, or a linear or branched alkyl group; R₁₄ represents a benzoyl group, a benzyl group, a modified benzyl group, or a linear or branched alkyl group; R₁₅ represents an —OR₁₇ group (wherein R₁₇ represents a linear or branched alkyl group or an aromatic ring) or an amido group; R₁₆ represents a hydrogen atom, or a linear or branched alkyl group; and X₇ and X₈ each represent a halogen atom, and X₇ and X₈ may be the same or different from each other.

A method of treating a disease caused by the storage of cholesteryl ester in cells according to the present invention includes administering Compound (1) or Compound (2) above, or a pharmacologically acceptable salt thereof to a human or a vertebrate other than a human (such as a rat or a mouse). Furthermore, a method of treating a disease caused by the storage of cholesteryl ester in cells according to the present invention includes administering any one of Compounds (6) to (8) above or a pharmacologically acceptable salt thereof to a human or a vertebrate other than a human (such as a rat or a mouse).

Examples of the diseases caused by the storage of cholesteryl ester in cells include Wolman disease and cholesteryl ester storage diseases.

### BEST MODE FOR CARRYING OUT THE INVENTION

Embodiments of the present invention completed on the basis of the above findings will now be described in detail using examples. When no particular descriptions are made in the embodiments and the examples, methods described in typical protocol collections such as J. Sambrook, E. F. Fritsch & T. Maniatis (Ed.), Molecular cloning, a laboratory manual (3rd edition), Cold Spring Harbor Press, Cold Spring Harbor, New York (2001); F. M. Ausubel, R. Brent, R. E. Kingston, D. D. Moore, J. G. Seidman, J. A. Smith, and K. Struhl (Ed.), Current Protocols in Molecular Biology, John Wiley & Sons Ltd. or methods obtained by modifying or changing the above-described methods are employed. When a commercially available reagent kit or a commercially available measuring device is used, unless otherwise specified, protocols attached thereto are used.

The object, features, advantages, and ideas of the present invention are obvious from this description to those skilled in the art. Those skilled in the art can easily reproduce the present invention on the basis of this description. The following embodiments of the invention, the specific examples, and the like include preferred embodiments of the present invention and are described for the purpose of exemplification or explanation. Accordingly, the present invention is not limited thereto. It should be understood by those skilled in the art that various changes and modifications may be made on the basis of this description without departing from the spirit and scope of the invention disclosed in this description.

### ==Pharmacological Effects of Cyclic Isodityrosine Derivatives of the Present Invention==

Compounds represented by the general formula (II) or (III), Compound (3), and pharmacologically acceptable salts thereof have an imipenem activity-potentiating effect as described in the examples below. Accordingly, when any of these compounds and the salts is administered with imipenem, this administration is useful for the treatment (including the prevention, the suppression, and the cure) of an infectious disease by bacteria such as MRSA, Gram-negative bacteria, or Gram-positive bacteria. It is known that imipenem does not have an effect on MRSA. Therefore, this administration is particularly useful for the treatment (including the prevention, the suppression, and the cure) of MRSA infections such as encephalitis, pneumonia, septicemia, peritonitis, enteritis, osteomyelitis, cholangitis, purulent skin lesions, decubitus ulcer infections, and food poisoning and toxic shock syndrome that are caused by a toxin produced by MRSA (such as enterotoxin or TSST-1). Similarly, it is considered that this administration is also useful for the treatment of Gram-positive bacterial infections or Gram-negative bacterial infections, for example, toxicosis, periodontitis, inflammatory diseases, vasculitis, IV-type allergic disease, staphylococcal scalded skin syndrome, Ritter's disease, impetigo bullosa of newborns, tumors, pneumonia, arthritis, meningitis, various purulent diseases, enteritis, meningitis, bacteremia, ocular infections, food poisoning, respiratory tract infections, otitis media, sinusitis, pharyngitis, scarlet fever, acute glomerulonephritis, rheumatic fever, impetigo, fulminant infections, tooth decay, urinary tract infections, wound infections, biliary tract infections, and aggravation of atopic diseases (such as atopic dermatitis) due to bacterial infection.

Accordingly, any of compounds represented by the general formula (II) or (III), Compound (3), or pharmacologically acceptable salts thereof can be used as an agent or a pharmaceutical composition for potentiating imipenem activity. In addition, an agent or a pharmaceutical composition containing both imipenem and any of the Compounds or a pharmacologically acceptable salt thereof can be used as an antibacterial agent or an antibiotic, and is effective for treating MRSA infections in particular. In this case, the above two ingredients of the agent or the pharmaceutical composition may be formed into separate dosage forms or a single dosage form as long as the two ingredients are added or administered so as to act in combination with each other. When the two ingredients are allowed “to act in combination with each other” by adding or administering both of the two ingredients, a synergistic effect of the two ingredients can be exerted. For example, a MRSA-growth inhibition activity, which is not exerted when each the two ingredients is separately used, can be exerted as described in the Examples below. Each of the two ingredients may be added or administered at the same time, as well as one after the other.

As described in the Examples below, it was found that compounds represented by the general formula (IV) and pharmacologically acceptable salts thereof have an effect of inhibiting the production of a cholesteryl ester in macrophages. When macrophages store a large amount of a cholesteryl ester, they become foam cells forming a fatty plaque or a fatty streak, which is an initial lesion of arteriosclerosis. In addition, the storage of cholesteryl ester in cells leads to the development of Wolman disease, cholesteryl ester storage diseases, and the like. Accordingly, compounds represented by the general formula (IV) and pharmacologically acceptable salts thereof are useful for the treatment (including the prevention, the suppression, and the cure) of diseases caused by the storage of cholesteryl ester in cells, for example, Wolman disease and cholesteryl ester storage diseases (such as arteriosclerosis (in particular, atherosclerosis)) and can be used as an agent or a pharmaceutical composition therefor.

Furthermore, a cholesteryl ester is formed into free cholesterol by an action of an acidic lipase and is pooled in cells. Therefore, it is considered that when the synthesis of a cholesteryl ester from oleic acid is suppressed, the production of cholesterol is also suppressed. Accordingly, compounds represented by the general formula (IV) and pharmacologically acceptable salts thereof have an effect of suppressing the production of cholesterol in macrophages. Therefore, compounds represented by the general formula (IV) and pharmacologically acceptable salts thereof are useful for the treatment (including the prevention, the suppression, and the cure) of diseases caused by an increase in cholesterol, for example, arteriosclerosis, hyperlipidemia, and coronary artery diseases (such as ischemic heart diseases, e.g., angina pectoris and myocardial infarction, and cerebrovascular diseases, e.g., cerebral apoplexy) and can be used as agents or pharmaceutical compositions therefor.

### ==Method of Manufacturing Cyclic Isodityrosine Derivatives of the Present Invention==

The cyclic isodityrosine derivatives of the present invention can be manufactured, for example, in accordance with the methods described in documents (such as Tetrahedron Letters 44 (2003) 7949-7952 and Tetrahedron Letters 60 (2004) 5623-5634) or the methods described in the examples below. An example of a method of manufacturing a cyclic isodityrosine derivative of the present invention will be described using reaction step formulae below. In the reaction step formulae, R₁₈ represents a hydrogen atom, a hydroxyl group, an —OR₂₂ group (wherein R₂₂ represents a linear or branched alkyl group or an aromatic ring), or an amido group; R₁₉ represents a hydrogen atom, a benzoyl group, a benzyl group, a modified benzyl group, or a linear or branched alkyl group; and R₂₀ represents a hydrogen atom, or a linear or branched alkyl group. In the reaction step formulae, R₂₁ represents a protective group of the amino group (e.g., a Boc (tert-butoxycarbonyl) group, a Cbz (benzyloxycarbonyl) group, an Fmoc group, a BocNH(CH₂)₅CO— group, or a BocNHCH(CH₂C₆H₅)CO— group); and X₉ and X₁₀ each represent a halogen atom, and X₉ and X₁₀ may be the same as or different from each other.

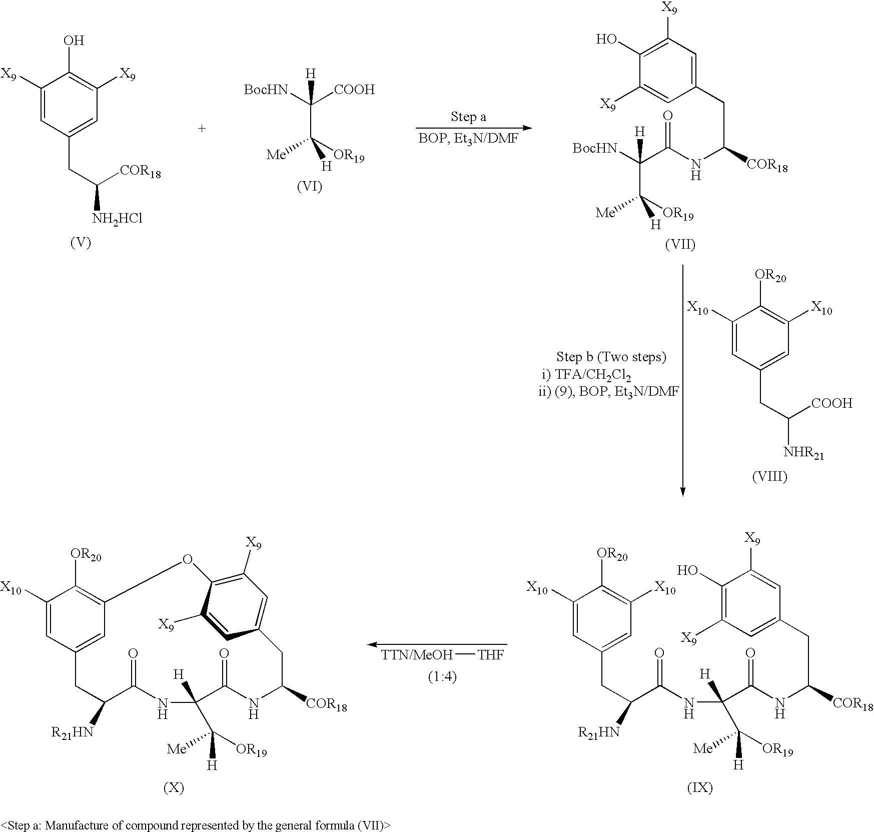

A compound represented by the general formula (V) a compound represented by the general formula (VI), a benzotriazole-1-yl-oxy-tris(dimethylamino) phosphonium hexafluorophosphate (BOP) reagent, and triethylamine (Et₃N) are dissolved in dimethylformamide (DMF), and the mixture is stirred. Subsequently, an aqueous solution of potassium hydrogensulfate is added to the mixture, and the mixture is then diluted with ethyl acetate. The mixture is then washed with a saturated saline solution. The organic layer is dehydrated over anhydrous sodium sulfate and then concentrated under reduced pressure to remove the solvent. The crude product thus obtained is purified by chromatography using, for example, silica gel, thereby allowing a compound represented by the general formula (VII) to be obtained.

### <Step b: Manufacture of Compound Represented by the General Formula (IX)>

The compound represented by the general formula (VII) is dissolved in trifluoroacetic acid-dichloromethane (TFA-CH₂Cl₂), and the mixture is then stirred. After the stirring, the mixture is concentrated under reduced pressure. The resulting precipitate is then dissolved in DMF containing a compound represented by the general formula (VIII), a BOP reagent, and Et₃N. After the resulting mixture is stirred, an aqueous solution of potassium hydrogensulfate is added to the mixture, and the mixture is diluted with ethyl acetate and then washed with a saturated saline solution. The organic layer is dehydrated over anhydrous sodium sulfate and then concentrated under reduced pressure to remove the solvent. The crude product thus obtained is purified by chromatography using, for example, silica gel, thereby allowing a compound represented by the general formula (IX) to be obtained.

### <Step c: Manufacture of Compound Represented by the General Formula (X)>

The compound represented by the general formula (IX) is added to a solvent (a mixed solution of anhydrous tetrahydrofuran (THF) and methanol (MeOH)) containing thallium (III) nitrate (TTN) (TTN/MeOH-THF (1:4)). After the mixture is stirred, sodium sulfite and water (one drop) are added thereto. Subsequently, the reaction mixture is subjected to celite filtration or the like, and is concentrated under reduced pressure, thus a crude product is obtained. The crude product is purified by chromatography using, for example, silica gel, thereby allowing a compound represented by the general formula (X) to be obtained.

A method of manufacturing some cyclic isodityrosine derivatives of the present invention has been described. The Boc group, the Cbz group, the Fmoc group, or the like of R₂₁ of the compound represented by general formula (X) may be removed. One of the hydrogen atoms of the resulting amino group produced by removing the Boc group, the Cbz group, the Fmoc group, or the like may then be substituted with an amido group, an acyl group, or a linear or branched alkyl group. These reactions are obvious to those skilled in the art and can be conducted by a known method. In addition, pharmacologically acceptable salts of the cyclic isodityrosine derivatives of the present invention, for example, alkali metal salts (e.g., sodium salts), alkaline earth metal salts (e.g., calcium salts), salts of other metals (e.g., aluminum salts), inorganic salts such as ammonium salts, and organic salts such as glucosamine salts are obvious to those skilled in the art and can be produced by a known method.

==Agents and Pharmaceuticals that Contain Cyclic Isodityrosine Derivative of the Present Invention==

A substance containing a cyclic isodityrosine derivative of the present invention or a pharmacologically acceptable salt thereof and/or imipenem as an active ingredient may be administered to a human or a vertebrate other than a human as pharmaceuticals, or may be used as a reagent for experiments. The pharmaceuticals containing a cyclic isodityrosine derivative of the present invention or a pharmacologically acceptable salt thereof as an active ingredient may be formed into dosage forms such as a tablet, a capsule, granules, a powder, or syrup and may be orally administered. These pharmaceuticals may be formed into dosage forms such as an injectable solution and a suppository and may be parenterally administered by injecting into the peritoneal cavity or a vein. The preparation of pharmaceuticals containing a cyclic isodityrosine derivative of the present invention or a pharmacologically acceptable salt thereof as an active ingredient can be performed by a known method using typical pharmaceutical additives (such as an excipient, a binder, a lubricant, a disintegrant, a taste corrigent, a smell corrigent, a solvent, and a stabilizer).

### EXAMPLES

The present invention will now be specifically described using examples. In the examples, the optical rotation was measured using a DIR-360 digital polarimeter with a sodium (D line) lamp manufactured by Jasco Corporation. The infrared (IR) absorption spectrum was measured with a Model A-202 spectrophotometer manufactured by Jasco Corporation. The nuclear magnetic resonance spectra (¹H-NMR and ¹³C-NMR, wherein CDCl₃ containing tetramethylsilane was used) were measured with a JNM-EX270 spectrometer and a JNM-GX400 spectrometer. The mass spectrum was measured with a JMS-700 (FAB) spectrometer manufactured by JEOL Ltd.

Silica gel thin-layer chromatography was performed using a kieselgel 60 PF₂₅₄ silica gel (manufactured by Merck Ltd.), and the coloration was performed with phosphomolybdic acid. Silica gel column chromatography was performed using a silica gel 60N (manufactured by Kanto Chemical Co., Inc.). The reactions were conducted in argon unless otherwise stated.

### Example 1

### Manufacture of N-Cbz-Thr(O-benzyl)-dibromo-iodo-eurypamide B methyl ester (Compound (1))

As in the method described in a document (Tetrahedron Letters 2004, 35, 8397-8400), L-tyrosine (manufactured by Junsei Chemical Co., Ltd.) was halogenated with bromine in methanol to prepare dibromo-L-tyrosine methyl ester. Similarly, N-Cbz-L-tyrosine methyl ester (manufactured by Kokusan Co., Ltd.) was halogenated with N-iodosuccinimide, and the resulting product was then hydrolyzed with sodium hydroxide, thereby allowing N-Cbz-diiodo-L-tyrosine to be prepared.

Subsequently, dibromo-L-tyrosine methyl ester (8.4 mmol), Boc-Thr(Bzl)-OH (8.4 mmol, manufactured by Watanabe Chemical Industries, Ltd.), a BOP reagent (8.35 mmol), and Et₃N (15 mmol) were dissolved in 17 mL of dimethylformamide (DMF), and the mixture was stirred for one night. After the stirring, a 5% aqueous solution of potassium hydrogensulfate was added to the mixture, and the mixed solution was then diluted with ethyl acetate and washed with a saturated saline solution. The organic layer was dehydrated over anhydrous sodium sulfate and then concentrated under reduced pressure to remove the solvent. The crude product thus obtained was purified by silica gel chromatography (hexane:ethyl acetate=1:1), thereby allowing a dipeptide to be prepared.

Subsequently, the dipeptide (0.94 mmol) was dissolved in trifluoroacetic acid-dichloromethane (TFA (2 mL)-CH₂Cl₂ 6 mL)), and the mixture was then stirred at 0° C. for three hours. After the stirring, the mixture was concentrated under reduced pressure. The precipitate was then added to DMF (0.6 mL) containing the N-Cbz-diiodo-L-tyrosine (0.94 mmol) prepared as above, a BOP reagent (0.94 mmol), and Et₃N (1.9 mmol) at 0° C. and dissolved. After the resulting mixture was stirred for one night, a 5% aqueous solution of potassium hydrogensulfate was added to the mixture, and the mixture was then diluted with ethyl acetate. The mixture was then washed with a saturated saline solution. The organic layer was dehydrated over anhydrous sodium sulfate and then concentrated under reduced pressure to remove the solvent. The crude product thus obtained was purified by silica gel chromatography (hexane:ethyl acetate=2:3), thereby allowing a tripeptide to be prepared.

The tripeptide (0.34 mmol) was dissolved in a solvent (140 mL of THF and 35 mL of methanol) containing TTN (1.0 mmol) at 0° C. After the mixture was stirred for one hour, sodium sulfite and water (one drop) were added thereto. Subsequently, the reaction mixture was subjected to celite filtration and was concentrated under reduced pressure. The crude product thus obtained was purified by silica gel chromatography (hexane:ethyl acetate=1:2), thereby allowing Compound (1) to be obtained.

¹HNMR (270 MHz, CDCl₃) δ 1.02 (3H, d, J=6.3 Hz), 2.31 (1H, t, J=12.5 Hz), 2.76 (1H, d, J=11.9 Hz), 3.13 (1H, dd, J=4.3, 14.2 Hz), 3.30 (1H, dd, J=4.1, 13.0 Hz), 3.37 (1H, m), 3.80 (3H, s), 4.42 (1H, dd, J=3.6, 6.6 Hz), 4.58 (1H, m), 4.68 (1H, d, J=12.4 Hz), 4.83 (1H, d, J=12.2 Hz), 4.92 (1H, m), 5.11 (1H, d, J=12.5 Hz), 5.23 (1H, d, J=12.2 Hz), 5.69 (1H, d, J=7.6 Hz), 5.75 (1H, d, J=1.6 Hz), 6.24 (1H, s), 6.66 (1H, d, J=6.9 Hz), 6.88 (1H, d, J=2.0 Hz), 7.07 (1H, d, J=1.6 Hz), 7.26-7.47 (10H, m), 7.52 (1H, d, J=9.9 Hz), and 7.63 (1H, d, J=1.6 Hz).

### Example 2

### Manufacture of N-Boc-Thr(O-benzyl)-dibromo-iodo-eurypamide B methyl ester (Compound (2))

Compound (2) was manufactured by the same method as that described in Example 1 except that N-Cbz-diiodo-L-tyrosine was changed to N-Boc-diiodo-L-tyrosine. N-Boc-diiodo-L-tyrosine was also manufactured, in accordance with the method described in the above document (Tetrahedron Letters 2004, 35, 8397-8400), by halogenating N-Boc-L-tyrosine methyl ester (manufactured by Kokusan Co., Ltd.) with N-iodosuccinimide, and then hydrolyzing the resulting product with sodium hydroxide.

¹HNMR (270 MHz, CDCl₃) δ1.01 (3H, d, J=6.3 Hz), 1.50 (9H, s), 2.35 (1H, t, J=12.5 Hz), 2.71 (1H, d, J=12.7 Hz), 3.21 (1H, dd, J=4.6, 14.3 Hz), 3.33 (1H, dd, J=4.0, 13.0 Hz), 3.79 (1H, m), 3.80 (3H, s), 4.45 (1H, dd, J=3.5, 6.8 Hz), 4.54 (1H, m), 4.64 (1H, d, J=14.2 Hz), 4.75 (1H, d, J=14.3 Hz), 4.94 (1H, m), 5.42 (1H, d, J=7.3 Hz), 5.72 (1H, s), 6.53 (1H, s), 6.79 (1H, d, J=6.9 Hz), 6.92 (1H, s), 7.07 (1H, s), 7.14-7.47 (5H, m), 7.52 (1H, d, J=9.7 Hz), and 7.72 (1H, s).

### Example 3

### Manufacture of N-Boc-dibromo-iodo-eurypamide B (Compound (3))

N-Boc-dibromo-iodo-eurypamide B methyl ester (Compound (12)) was manufactured by the same method as that described in Example 1 except that N-Cbz-diiodo-L-tyrosine was changed to N-Boc-diiodo-L-tyrosine and that Boc-Thy(Bzl)-OH was changed to N-Boc-L-Thr (manufactured by Watanabe Chemical Industries, Ltd.).

¹HNMR (270 MHz, CD₃OD) δ1.07 (3H, d, J=6.3 Hz), 1.50 (9H, s), 2.56 (1H, t, J=12.7 Hz), 2.73 (1H, d, J=14.7 Hz), 3.23 (1H, dd, J=13.9, 5.1 Hz), 3.38 (1H, dd, J=13.2, 4.4 Hz), 3.86 (3H, s), 4.10 (1H, m), 4.3.5 (1H, m), 4.42 (1H, m), 4.99 (1H, m), 5.39 (1H, d, J=7.1 Hz), 5.79 (1H, d, J=1.5 Hz), 6.11 (1H, s), 7.09 (1H, d, J=2.0 Hz), 7.30 (1H, d, J=1.5 Hz), 7.47 (1H, d, J=9.8 Hz), and 7.63 (1H, d, J=2.0 Hz).

Subsequently, N-Boc-dibromo-iodo-eurypamide B methyl ester (0.03 mmol) was dissolved in MeOH (0.5 mL)-1 M NaOH (0.5 mL) at 0° C., and the solution was stirred for 30 minutes. The solution was then treated with Amberlite IR 120B(H⁺), and the resulting solution was concentrated under reduced pressure. The crude product thus obtained was purified by silica gel chromatography (chloroform:methanol=5:1), thereby allowing Compound (3) to be obtained.

¹HNMR (270 MHz, CD₃OD) δ1.09 (3H, d, J=6.6 Hz), 1.49 (9H, s), 2.56 (1H, m), 2.88 (1H, m), 3.05 (1H, m), 3.38 (1H, m), 4.16 (1H, m), 4.40 (1H, m), 4.63 (2H, m), 5.77 (1H, d, J=1.8 Hz), 6.77 (1H, d, J=8.7 Hz), 7.00 (1H, s), 7.42 (1H, d, J=1.7 Hz), 7.64 (1H, d, J=8.9 Hz), 7.70 (1H, d, J=1.8 Hz), 7.92 (1H, d, J=8.7 Hz), 7.98 (1H, d, J=3.1 Hz), and 8.04 (1H, d, J=8.7 Hz).

### Example 4

### Manufacture of dibromo-iodo-eurypamide B methyl ester (Compound (4))

N-Boc-dibromo-iodo-eurypamide B methyl ester (0.03 mmol) was dissolved in CH₂Cl₂ (1 mL)-TFA (1 mL), and the solution was stirred at 0° C. for two hours. After the stirring, the solution was concentrated under reduced pressure. The crude product thus obtained was purified by silica gel chromatography (chloroform:methanol=5:1), thereby allowing Compound (4) to be obtained.

¹HNMR (270 MHz, CD₃OD) δ 1.13 (3H, d, J=6.3 Hz), 2.65 (1H, t, J=12.7 Hz), 2.92 (1H, dd, J=5.3, 15.1 Hz), 3.20 (1H, d, J=15.0 Hz), 3.40 (1H, dd, J=3.8, 13.1 Hz), 3.81 (3H, s), 4.12 (2H, m), 4.41 (1H, d, J=2.8 Hz), 4.94 (1H, d, J=3.8 Hz), 5.78 (1H, d, J=1.8 Hz), 7.26 (1H, d, J=1.8 Hz), 7.46 (1H, d, J=1.8 Hz), and 7.73 (1H, d, J=1.6 Hz).

### Example 5

### Manufacture of Thr(O-benzyl)-dibromo-iodo-eurypamide B methyl ester (Compound (5))

Compound (2) (0.03 mmol) obtained in Example 2 was dissolved in CH₂Cl₂ (1 mL)-TFA (1 mL), and the solution was stirred at 0° C. for two hours. After the stirring, the solution was concentrated under reduced pressure. The crude product thus obtained was purified by silica gel chromatography (chloroform:methanol=˜5:1), thereby allowing Compound (5) to be obtained.

¹HNMR (400 MHz, CD₃OD) δ 1.15 (3H, d, J=5.9 Hz), 2.61 (1H, t, J=12.7 Hz), 2.81 (1H, dd, J=5.4, 14.6 Hz), 3.06 (1H, dd, J=2.4, 14.6 Hz), 3.40 (1H, dd, J=3.9, 13.2 Hz), 3.73 (3H, s), 3.89 (1H, dd, J=2.6, 5.1 Hz), 4.03 (1H, dd, J=2.6, 6.1 Hz), 4.51 (1H, d, J=11.7 Hz), 4.53 (1H, d, J=2.4 Hz), 4.62 (1H, d, J=11.2 Hz), 4.95 (1H, m), 5.74 (1H, d, J=2.0 Hz), 7.25 (1H, d, J=2.0 Hz), 7.27-7.31 (5H, m), 7.38 (1H, d, J=2.0 Hz), and 7.72 (1H, d, J=2.0 Hz).

### Example 6

### Manufacture of Compound (6)

The dipeptide (1 mmol) obtained in Example 1 was dissolved in trifluoroacetic acid-dichloromethane (TFA (2 mL)-CH₂Cl₂ (4 mL)), and the solution was then stirred at 0° C. for three hours. After the stirring, the solution was concentrated under reduced pressure. The precipitate was then added to acetonitrile (50 mL) containing Fmoc-Tyr(3,5-I₂)-OH (1 mmol) (M00280, manufactured by Watanabe Chemical Industries, Ltd.), a BOP reagent (1 mmol), and Et₃N (0.5 mL) at 0° C. and dissolved. After the resulting mixture was stirred for one night, the mixture was concentrated under reduced pressure to remove the solvent. The crude product thus obtained was purified by silica gel chromatography (chloroform:methanol 100:3), thereby allowing a tripeptide to be prepared.

The tripeptide (0.05 mmol) was dissolved in a solvent (20 mL of THF and 5 mL of methanol) containing TTN (0.1 mmol) at 0° C. After the mixture was stirred for one hour, sodium sulfite was added thereto. Subsequently, the reaction mixture was subjected to celite filtration and was concentrated under reduced pressure. The crude product thus obtained was purified by silica gel chromatography (chloroform:ethyl acetate=5:1), thereby allowing Compound (6) to be obtained.

¹HNMR (270 MHz, CD₃OD) δ 1.09 (3H, d, J=6.3 Hz), 2.33 (1H, t, J=12.4 Hz), 2.81 (1H, d, J=13.7 Hz), 3.15 (1H, dd, J=14.6, 5.9 Hz), 3.31 (1H, dd, J=13.2, 4.4 Hz), 3.83 (3H, s), 4.27 (3H, m), 4.42 (1H, m), 4.50 (1H, dd, J=8.5, 6.1 Hz), 4.58 (1H, m), 4.71 (1H, d, J=12.2 Hz), 4.86 (1H, d, J=12.2 Hz), 4.93 (1H, m), 5.75 (1H, d, J=7.3 Hz), 5.84 (1H, s), 6.19 (1H, brs), 6.46 (1H, d, J=6.8 Hz), 6.90 (1H, s), 7.18 (1H, s), 7.29-7.69 (13H, m), and 7.77 (2H, m).

### Example 7

### Manufacture of Compound (7)

An acetonitrile (2 mL) solution containing Compound (5) (153 mg, 0.18 mmol) obtained in Example 5 was added with N-Boc-6-aminohexanoic acid (46 mg, 0.20 mmol, 05126-62 manufactured by Nacalai Tesque, Inc.), a BOP reagent (88 mg, 0.20 mmol) and Et₃N (100 μL), and stirred in the range of 0° C. to room temperature for 16 hours. After the stirring, the mixed solution was diluted with ethyl acetate and then washed with a saturated saline solution. The organic layer was dehydrated over anhydrous sodium sulfate and then concentrated under reduced pressure to remove the solvent. The crude product thus obtained was purified by silica gel chromatography (hexane:ethyl acetate=2:1), thereby allowing 148.3 mg of Compound (7) to be obtained.

¹HNMR (270 MHz, CD₃OD) δ1.14 (3H, d, J=6.3 Hz), 1.37 (2H, m), 1.42 (9H, s), 1.53 (2H, m), 1.66 (2H, m), 2.22 (2H, m), 2.60 (1H, t, J=12.7 Hz), 2.87 (1H, d, J=15.6 Hz), 3.01 (3H, m), 3.39 (1H, dd, J=12.9, 3.7 Hz), 3.74 (3H, s), 3.98 (1H, dd, J=6.3, 2.9 Hz), 4.51 (1H, d, J=13.2 Hz), 4.52 (1H, s), 4.62 (1H, d, J=11.7 Hz), 4.68 (1H, d, J=2.9 Hz), 4.70 (1H, m), 5.73 (1H, d, J=1.5 Hz), 7.00 (1H, s), 7.26-7.31 (5H, m), 7.38 (1H, s), and 7.72 (1H, d, J=2.0 Hz).

### Example 8

### Manufacture of Compound (8)

An acetonitrile (1 mL) solution containing Compound (5) (0.069 mmol) obtained in Example 5 was added with Boc-Phe-OH (19 mg, 0.072 mmol, B-5394 manufactured by Sigma Corporation), a BOP reagent (0.072 mmol) and Et₃N (0.1 mL), and stirred at 0° C. for 16 hours. After the stirring, the mixed solution was concentrated under reduced pressure to remove the solvent. The crude product thus obtained was purified by silica gel chromatography (chloroform:methanol=20:1), thereby allowing Compound (7) to be obtained.

¹HNMR (270 MHz, CDCl₃) δ1.07 (3H, d, J=6.4 Hz), 1.38 (9H, s), 2.35 (1H, t, J=12.4 Hz), 2.75 (1H, d, J=14.7 Hz), 3.01 (1H, m), 3.22 (2H, m), 3.32 (1H, dd, J=13.0, 4.1 Hz), 3.78 (1H, m), 3.83 (3H, s), 4.36 (2H, m), 4.60 (1H, brs), 4.69 (1H, d, J=12.2 Hz), 4.83 (1H, d, J=12.0 Hz), 4.93 (1H, m), 5.78 (1H, d, J=1.6 Hz), 6.13 (1H, s), 6.43 (1H, d, J=6.9 Hz), 6.83 (1H, d, J=6.6 Hz), 6.91 (1H, d, J=1.6 Hz), 6.95 (1H, s), 7.17-7.52 (6H, m), 7.44 (1H, s), and 7.63 (1H, d, J=1.8 Hz).

### Example 9

### Manufacture of Compound (9)

Compound (2) (0.03 mmol) obtained in Example 2 was dissolved in MeOH (0.5 mL)-1 M NaOH (0.5 mL) at 0° C., and the solution was stirred for 30 minutes. The solution was then treated with Amberlite IR 12.0B(H⁺), and the resulting solution was then concentrated under reduced pressure. The crude product thus obtained was purified by silica gel chromatography (chloroform:methanol=5:1), thereby allowing Compound (9) to be obtained.

¹HNMR (270 MHz, CD₃OD) δ1.13 (3H, d, J=5.9 Hz), 1.49 (9H, s), 2.59 (1H, t, J=12.5 Hz), 2.84 (1H, d, J=12.7 Hz), 3.03 (1H, dd, J=13.7, 6.1 Hz), 3.40 (1H, dd, J=10.3, 3.9 Hz), 4.01 (1H, m), 4.36 (1H, m), 4.51 (1H, m), 4.53 (1H, d, J=11.2 Hz), 4.62 (1H, d, J=12.2 Hz), 4.81 (1H, m), 5.74 (1H, s), 7.01 (1H, s), 7.24-7.35 (6H, m), 7.72 (1H, s), 8.08 (1H, d, J=9.3 Hz), and 8.26 (1H, d, J=9.8 Hz).

### Example 10

### Manufacture of Compound (10)

Compound (1) (0.07 mmol) obtained in Example 1 was dissolved in MeOH (1 mL)-1 M NaOH (0.4 mL) at 0° C., and the solution was stirred for three hours. The solution was neutralized by adding 1 N-HCl. The reaction solution was then diluted with ethyl acetate and washed with a saturated saline solution. The organic layer was dehydrated over anhydrous sodium sulfate and then concentrated under reduced pressure to remove the solvent. The crude product thus obtained was purified by silica gel chromatography (ethyl acetate:methanol=5:1), thereby allowing Compound (10) to be obtained.

¹HNMR (270 MHz, CD₃OD) δ 1.11 (3H, d, J=6.3 Hz), 2.58 (1H, t, J=12.7 Hz), 2.87 (1H, d, J=11.7 Hz), 2.96 (1H, dd, J=13.4, 5.6 Hz), 3.39 (1H, dd, J=12.9, 3.7 Hz), 3.96 (1H, m), 4.52 (3H, m), 4.61 (1H, d, J=11.7 Hz), 4.84 (1H, m), 5.04 (1H, d, J=12.7 Hz), 5.19 (1H, d, J=12.7 Hz), 5.75 (1H, s), 7.02 (1H, s), 7.25 (1H, d, J=2.0 Hz), 7.23-7.38 (10H, m), 7.70 (1H, s), 8.08 (1H, d, J=9.3 Hz), and 8.24 (1H, d, J=9.3 Hz).

### Example 11

### Manufacture of Compound (11)

Compound (9) (0.2 mmol) produced in Example 9 was dissolved in CH₂Cl₂ (2 mL)-TFA (0.5 mL), and the solution was stirred at 0° C. for two hours. After the stirring, the solution was concentrated under reduced pressure. The crude product thus obtained was purified by silica gel chromatography (ethyl acetate:methanol=5:1), thereby allowing Compound (11) to be obtained.

¹HNMR (270 MHz, CD₃OD) δ 1.06 (3H, d, J=6.3 Hz), 2.52 (1H, t, J=12.7 Hz), 2.84 (1H, dd, J=14.9, 4.9 Hz), 3.08 (1H, d, J=14.0 Hz), 3.33 (1H, dd, J=12.8, 3.7 Hz), 3.98 (1H, dd, J=6.3, 2.5 Hz), 4.05 (3H, m), 4.42 (1H, d, J=11.3 Hz), 4.47 (1H, m), 4.51 (1H, d, J=11.6 Hz), 4.76 (1H, m), 5.70 (1H, d, J=1.6 Hz), 7.17 (1H, s), 7.16-7.25 (5H, m), 7.28 (1H, d, J=1.6 Hz), 7.63 (1H, d, J=1.5 Hz), 8.15 (1H, d, J=9.4 Hz), and 8.25 (1H, d, J=9.7 Hz).

### Example 12

### Measurement of Imipenem Activity-Potentiating Effect

In this example, clinically isolated MRSA K24 strain was used as an assay strain. A plate for control was prepared by adding 1.5% of agar (manufactured by Shimizu Shokuhin Kaisha, Ltd.) to Mueller-Hinton Broth (manufactured by DIFCO Laboratories), and a plate for assay was prepared by adding imipenem (trade name: Tienam for intramuscular injection, titer: 0.5, manufactured by Banyu Pharmaceutical Co., Ltd.) to the above composition so that the final concentration of imipenem was 10 μg/mL. These plates were used as assay plates. An inoculum solution was prepared by culturing the assay strain in Mueller-Hinton Broth for one night, and diluting the culture solution with the same culture medium so as to have a concentration of 0.5 McFarland units (about 10⁸ cfu/mL). This inoculum solution was applied on each of the plates with a sterile cotton swab in accordance with the National Committee For Clinical Laboratory Standards (NCCLS) method. The antibacterial activities against the assay strain in the plates were evaluated by a paper-disc method as follows. Paper discs with a diameter of 6 mm impregnated with 10 μg of assay medical agents (eurypamides A, A′, B, and D, and Compounds (1) to (12)) were placed on each of the plates, and the plates were cultured at 37° C. for 20 hours.

On the plate for control, the assay strain was grown under the paper discs. This result showed that Compounds (1) to (12) in Table 1 did not have anti-MRSA activity by themselves. In addition, the assay strain could be grown on the plate for assay. Accordingly, when the concentration of imipenem was 10 μg/mL, anti-MRSA activity was not observed in imipenem. If the compound impregnated in the paper disc acts in combination with imipenem to exert anti-MRSA activity, growth of the assay strain would not be observed under and around the paper disc. Therefore, in this example, the imipenem activity-potentiating effect for MRSA is represented by a diameter of an inhibition circle (the diameter passing through the center of the paper disc) on the assay plates.

| Name of | MRSA (mm) | Macrophage |
|---|---|---|
| compound | [10 μg/6 mm disc] | [IC₅₀ μg/mL] |
| Eurypamide A | — | — |
| Eurypamide A′ | — | — |
| Eurypamide B | — | — |
| Eurypamide D | — | — |
| Compound (1) | 8 | 3 |
| Compound (2) | 8 | 3 |
| Compound (3) | 10 | — |
| Compound (4) | 8 | — |
| Compound (5) | 8 | — |
| Compound (6) | — | 0.32 |
| Compound (7) | — | 4.2 |
| Compound (8) | — | 5 |
| Compound (9) | 17 | — |
| Compound (10) | 23 | — |
| Compound (11) | 12 | — |
| Compound (12) | 9 | — |

According to the results as shown in Table 1, eurypamides A to D did not exhibit the imipenem activity-potentiating effect, whereas Compounds (1) to (5) and Compounds (9) to (12) exhibited the imipenem activity-potentiating effect in the range of 8 to 23 mm in this assay. That is, of Compounds (1) to (5) and Compounds (9) to (12) exerted anti-MRSA activity by acting in combination with imipenem.

### Example 13

### Effect of Inhibiting Production of Cholesteryl Ester in Mouse Peritoneal Macrophage

Production of a cholesteryl ester in mouse peritoneal macrophages was performed by partly modifying the method by Nishikawa et al (Nishikawa et al., the Journal of Biological Chemistry (JBC) 265, pp. 5226-5231, 1990). Mouse peritoneal macrophages were prepared from an ICR female mouse as follows. First, cells were isolated from the mouse peritoneal cavity using a Hank's balanced salt solution (HBSS) and then suspended in a GIT medium so as to have a concentration of 2×10⁶ cells/mL. Subsequently, 0.25 mL of the suspension was placed in each of 48 wells on a plastic culture plate, and the plate was cultured in the presence of 5% CO₂ at 37° C. for two hours. In order to remove non-adhesive cells, each well was then washed with 0.25 mL of an HBSS three times. Finally, 0.25 mL of a culture medium A (Dulbecco's modified Eagle's medium (DMEM) containing 100 u/mL of penicillin, 100 μg/mL of streptomycin, and an 8% lipoprotein-free serum) was added thereto.

The effect of inhibiting the synthesis of a cholesteryl ester of the agents (eurypamides A, A′, B, and D, and Compounds (1) to (12)) in the macrophages thus prepared was evaluated as follows: The amount of [¹⁴C] cholesteryl ester and [¹⁴C] triacylglycerol synthesized from [¹⁴C] oleic acid was measured as follows, and a concentration of the agent at which the synthesis was inhibited by 50% (IC₅₀: μg/mL) was determined.

First, the macrophages (5×10⁵ cells/0.25 mL/well) were cultured in a plastic culture plate with 48 wells. Subsequently, 5 μL of [¹⁴C] oleic acid (1 nmol, 0.05 μCi, which was dissolved in a phosphate buffered saline (PBS) containing 10% ethanol), liposome (having a composition of phosphatidylcholine/phosphatidylserine/diacetylphosphate/cholesterol=10/10/2/5 (nmol) in 10 μL of 0.3 M glucose), and 2.5 μL of each agent dissolved in methanol were added to each well. The plate was cultured for 14 hours, and the culture medium was then removed. Each of the wells was washed with a PBS three times. Subsequently, 0.25 mL of a PBS containing 0.1% (w/v) sodium dodecyl sulfate (SDS) was added to each well to lyse the cells. Lipid in the cells was extracted by the method by Bligh & Dyer (Bligh & Dyer, Can. J. Biochem. Physiol., 37, pp. 911-917, 1959). The volume of the organic solvent was reduced by centrifugation in vacuum. The solution was spotted on a thin-layer chromatography (TLC) plate (silica gel F245, thickness: 0.5 mm, manufactured by Merck Ltd.) and developed with hexane/diethyl ether/acetic acid (70/30/1, v/v). The amounts of separated [¹⁴C] cholesteryl oleate and [¹⁴C] triacylglycerol were analyzed with a bioimage analyzer (BAS2000 manufactured by FUJIFILM Corporation). The results are shown in Table 1 above. The symbol “-” in the column of macrophage in Table 1 means that the compound did not exhibit inhibition in its amount of 10 μg/mL.

As shown in Table 1, only in the cases of Compounds (1), (2), (6), (7), and (8), activity of inhibiting the production of cholesteryl oleate was detected, and the IC₅₀ thereof was 3 μg/mL, 3 μg/mL, 0.32 μg/mL, 4.2 μg/mL, and 5 μg/mL, respectively. Thus, it was found that Compounds (1) and (2), and Compounds (6) to (8) have an effect of inhibiting the synthesis of a cholesteryl ester in mouse peritoneal macrophages.

### INDUSTRIAL APPLICABILITY

The present invention can provide a novel cyclic isodityrosine derivative having a physiological effect, a pharmacologically acceptable salt thereof, an agent and a pharmaceutical composition that contain the cyclic isodityrosine derivative or a pharmacologically acceptable salt thereof as an active ingredient, and a method of using the above.

1. A compound represented by general formula (1) or a pharmacologically acceptable salt thereof: 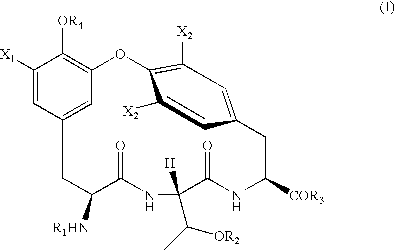
wherein R₁ represents a hydrogen atom, an amido group, a Boc group, a Cbz group, an Fmoc group, a BocNH(CH₂)₅CO— group, a BocNHCH(CH₂C₆H₅)CO— group, or a linear or branched alkyl group; R₂ represents a benzoyl group, a benzyl group, a modified benzyl group, or a linear or branched alkyl group; R₃ represents a hydrogen group, an —OR₅ group, wherein R₅ represents a linear or branched alkyl group or an aromatic ring, or an amido group; R₄ represents a hydrogen atom, or a linear or branched alkyl group; and X₁ and X₂ each represent a halogen atom, wherein X₁ and X₂ may be the same as or different from each other.

2. A compound represented by any one of formulae (1) to (5) or a pharmacologically acceptable salt thereof:

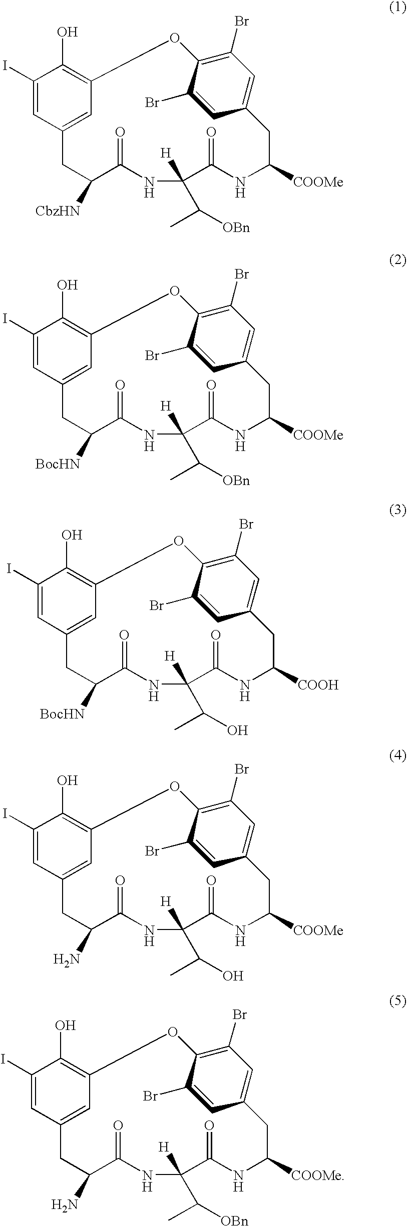

3. A compound represented by any one of formulae (6) to (8) or a pharmacologically acceptable salt thereof:

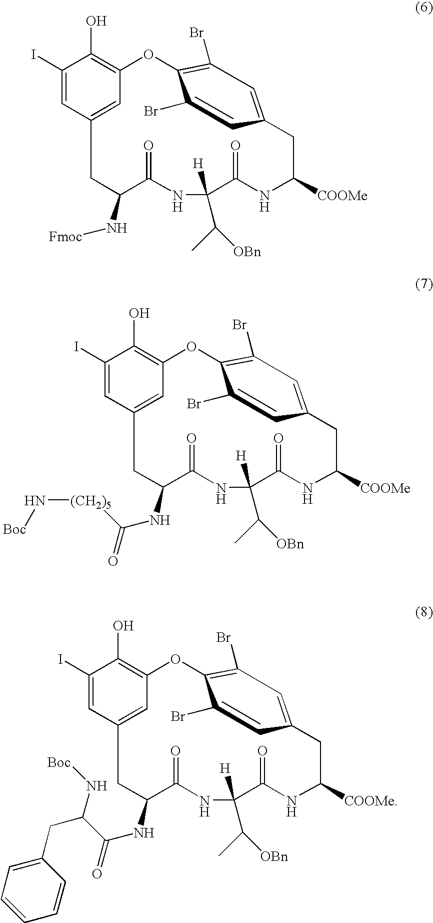

4. A compound represented by general formula (II) or a pharmacologically acceptable salt thereof: 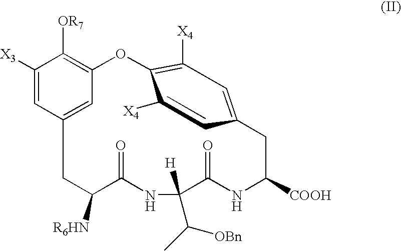
wherein R₆ represents a hydrogen atom, a Boc group, or a Cbz group; R₇ represents a hydrogen atom, or a linear or branched alkyl group; and X₃ and X₄ each represent a halogen atom, wherein X₃ and X₄ may be the same as or different from each other.

5. A compound represented by any one of formulae (9) to (11) or a pharmacologically acceptable salt thereof:

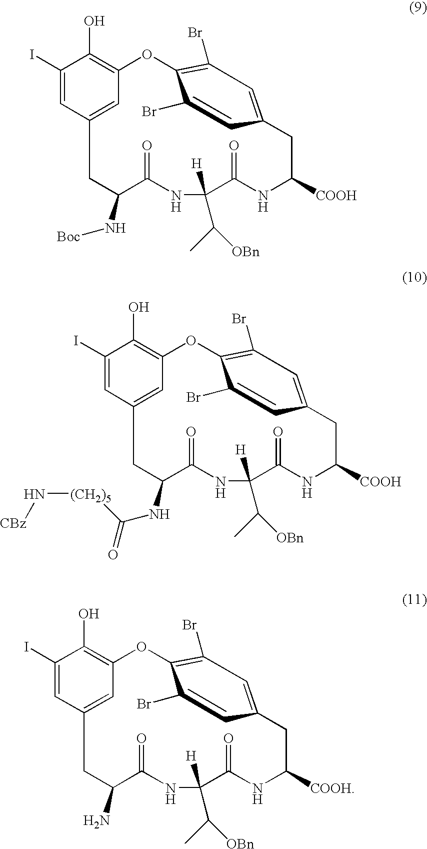

6-24. (canceled)

25. A pharmaceutical composition, comprising a compound represented by general formula (III) or a pharmacologically acceptable salt thereof, and imipenem as active ingredients: 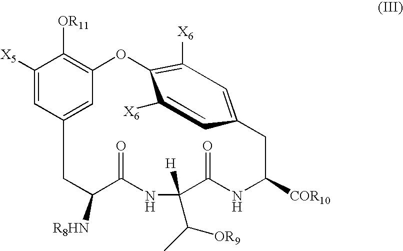
wherein R₈ represents a hydrogen atom, an amido group, a Boc group, a Cbz group, or a linear or branched alkyl group; R₉ represents a hydrogen atom, a benzoyl group, a benzyl group, a modified benzyl group, or a linear or branched alkyl group; R₁₀ represents a hydrogen atom, an —OR₁₂ group, wherein R₁₂ represents a linear or branched alkyl group or an aromatic ring, or an amido group; R₁₁ represents a hydrogen atom, or a linear or branched alkyl group; X₅ and X₆ each represent a halogen atom, wherein X₅ and X₆ may be the same as or different from each other; but when R₉ is a hydrogen atom, R₈ is a hydrogen atom or a Boc group.

26. A pharmaceutical composition, comprising a compound represented by any one of formulae (1) to (5) or a pharmacologically acceptable salt thereof, and imipenem as active ingredients:

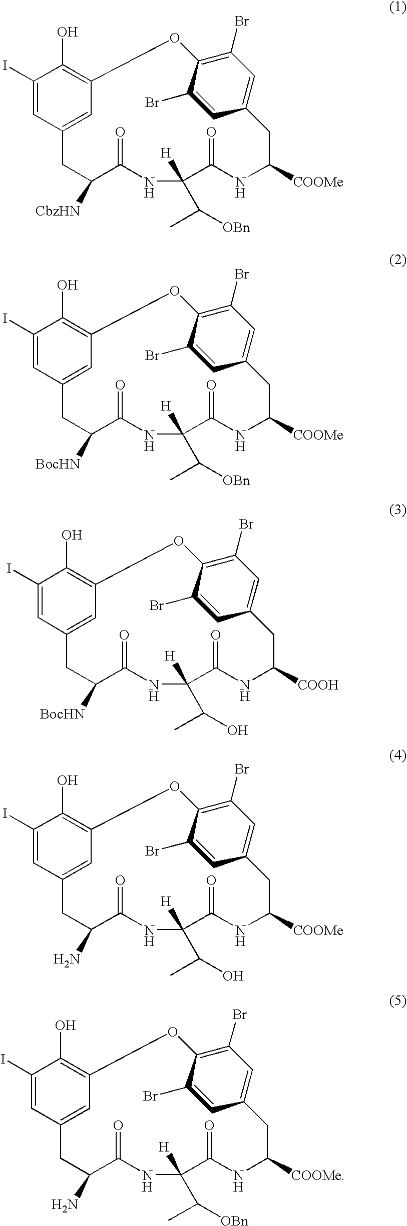

27. A pharmaceutical composition, comprising a compound represented by formulae (12) or a pharmacologically acceptable salt thereof, and imipenem as active ingredients:

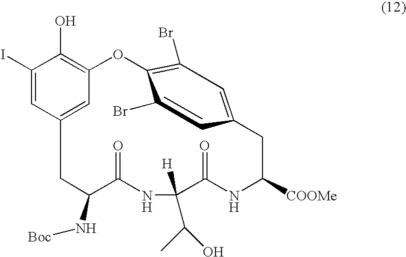

28. A pharmaceutical composition, comprising a compound represented by general formula (II) or a pharmacologically acceptable salt thereof, and imipenem as active ingredients: 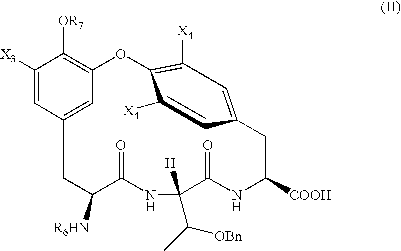
wherein R₆ represents a hydrogen atom, a Boc group, or a Cbz group; R₇ represents a hydrogen atom, or a linear or branched alkyl group; and X₃ and X₄ each represent a halogen atom, wherein X₃ and X₄ may be the same as or different from each other.

29. A pharmaceutical composition, comprising a compound represented by any one of formulae (9) to (11) or a pharmacologically acceptable salt thereof, and imipenem as active ingredients:

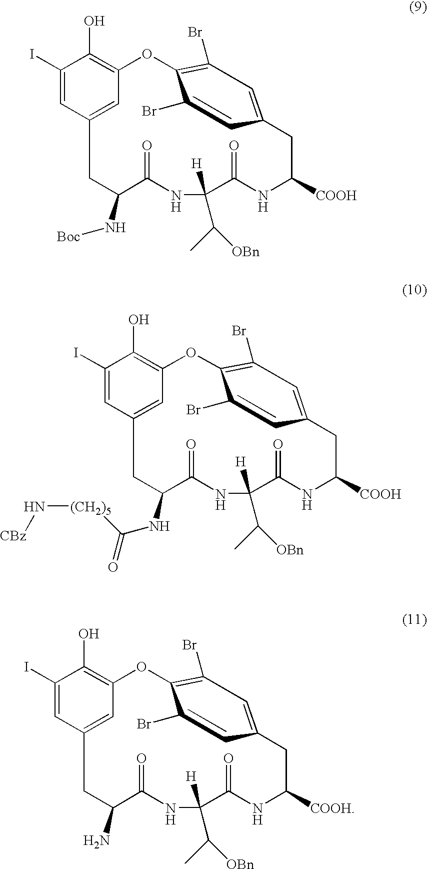

30. The pharmaceutical composition according to claim 25 wherein the bacteria is methicillin-resistant Staphylococcus aureus (MRSA).

31. The pharmaceutical composition according to claim 30, wherein the disease is at least one disease selected from encephalitis, pneumonia, septicemia, peritonitis, enteritis, osteomyelitis, cholangitis, purulent skin lesions, decubitus ulcer infection, and food poisoning and toxic shock syndrome that are caused by a toxin produced by MRSA.

32-46. (canceled)

47. A method of potentiating activity of imipenem, comprising:
allowing a compound represented by general formula (III) or a pharmacologically acceptable salt thereof to act in combination with the imipenem: 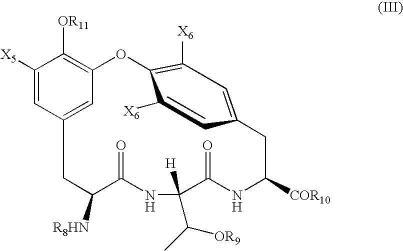
wherein R₈ represents a hydrogen atom, an amido group, a Boc group, a Cbz group, or a linear or branched alkyl group; R₉ represents a hydrogen atom, a benzoyl group, a benzyl group, a modified benzyl group, or a linear or branched alkyl group; R₁₀ represents a hydrogen atom, an —OR₁₂ group, wherein R₁₂ represents a linear or branched alkyl group or an aromatic ring, or an amido group; R₁₁ represents a hydrogen atom, or a linear or branched alkyl group; X₅ and X₆ each represent a halogen atom, wherein X₅ and X₆ may be the same as or different from each other; but when R₉ is a hydrogen atom, R₈ is a hydrogen atom or a Boc group.

48. A method of potentiating activity of imipenem, comprising:
allowing a compound represented by any one of formulae (1) to (5) or a pharmacologically acceptable salt thereof to act in combination with the imipenem:

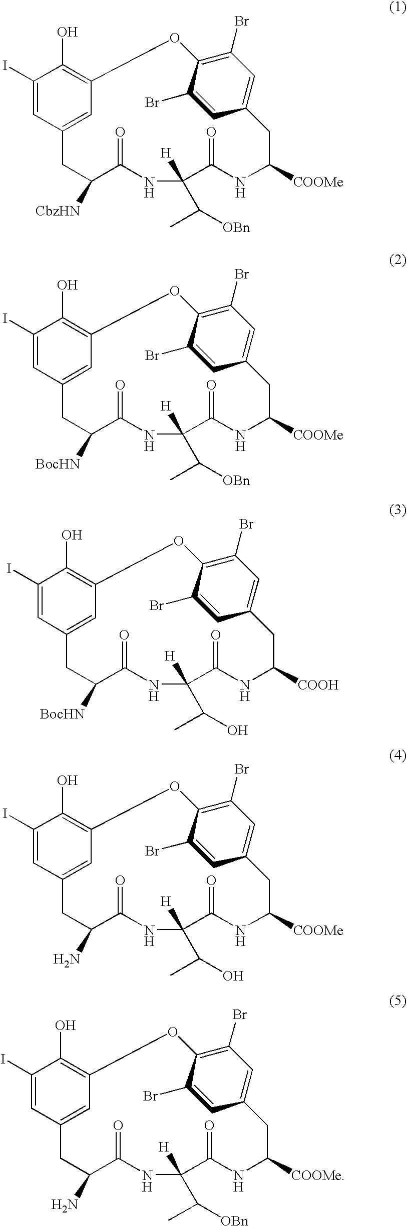

49. A method of potentiating activity of imipenem, comprising:
allowing a compound represented by formula (12) or a pharmacologically acceptable salt thereof to act in combination with the imipenem:

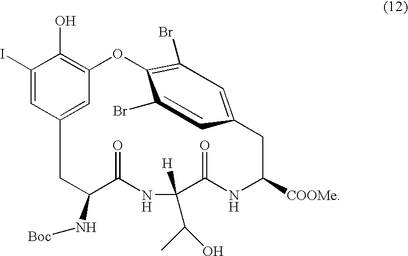

50. A method of potentiating activity of imipenem, comprising:
allowing a compound represented by general formula (II) or a pharmacologically acceptable salt thereof to act in combination with the imipenem: 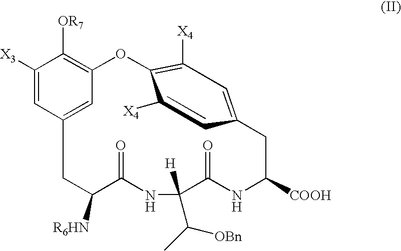
wherein R₆ represents a hydrogen atom, a Boc group, or a Cbz group; R₇ represents a hydrogen atom, or a linear or branched alkyl group; and X₃ and X₄ each represent a halogen atom, wherein X₃ and X₄ may be the same as or different from each other.

51. A method of potentiating activity of imipenem, comprising:
allowing a compound represented by any one of formulae (9) to (11) or a pharmacologically acceptable salt thereof to act in combination with the imipenem:

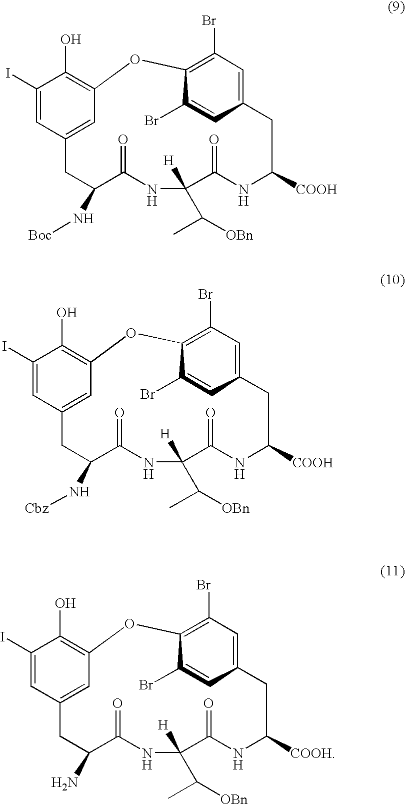

52. The method of potentiating activity of imipenem according to claim 47, wherein the activity of the imipenem is antibacterial activity.

53. The method of potentiating activity of imipenem according to claim 52, wherein the antibacterial activity is antibacterial activity against methicillin-resistant Staphylococcus aureus (MRSA).

54. A method of potentiating activity of a cyclic isodityrosine derivative represented by general formula (III) or a pharmacologically acceptable salt thereof, comprising:
allowing imipenem to act in combination with the cyclic isodityrosine derivative: 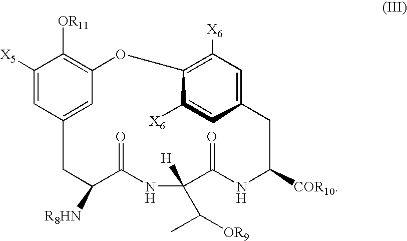
wherein R₈ represents a hydrogen atom, an amido group, a Boc group, a Cbz group, or a linear or branched alkyl group; R₉ represents a hydrogen atom, a benzoyl group, a benzyl group, a modified benzyl group, or a linear or branched alkyl group; R₁₀ represents a hydrogen atom, an —OR₁₂ group, wherein R₁₂ represents a linear or branched alkyl group or an aromatic ring, or an amido group; R₁₁ represents a hydrogen atom, or a linear or branched alkyl group; X₅ and X₆ each represent a halogen atom, wherein X₅ and X₆ may be the same as or different from each other; but when R₉ is a hydrogen atom, R₈ is a hydrogen atom or a Boc group.

55. A method of potentiating activity of a cyclic isodityrosine derivative represented by any one of formulae (1) to (5) or a pharmacologically acceptable salt thereof, comprising:
allowing imipenem to act in combination with the cyclic isodityrosine derivative:

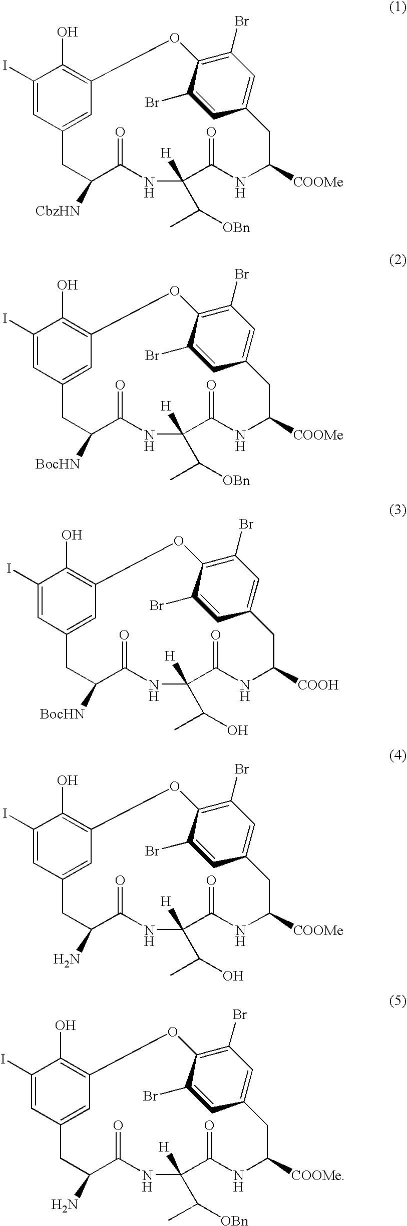

56. A method of potentiating activity of a cyclic isodityrosine derivative represented by formula (12) or a pharmacologically acceptable salt thereof, comprising:
allowing imipenem to act in combination with the cyclic isodityrosine derivative:

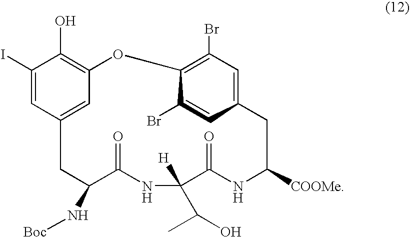

57. A method of potentiating activity of a cyclic isodityrosine derivative represented by general formula (II) or a pharmacologically acceptable salt thereof, comprising:
allowing imipenem to act in combination with the cyclic isodityrosine derivative: 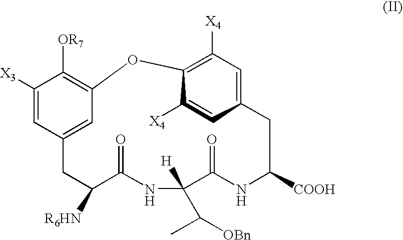
wherein R₆ represents a hydrogen atom, a Boc group, or a Cbz group; R₇ represents a hydrogen atom, or a linear or branched alkyl group; and X₃ and X₄ each represent a halogen atom, wherein X₃ and X₄ may be the same as or different from each other.

58. A method of potentiating activity of a cyclic isodityrosine derivative represented by any one of formulae (9) to (11) or a pharmacologically acceptable salt thereof, comprising:
allowing imipenem to act in combination with the cyclic isodityrosine derivative:

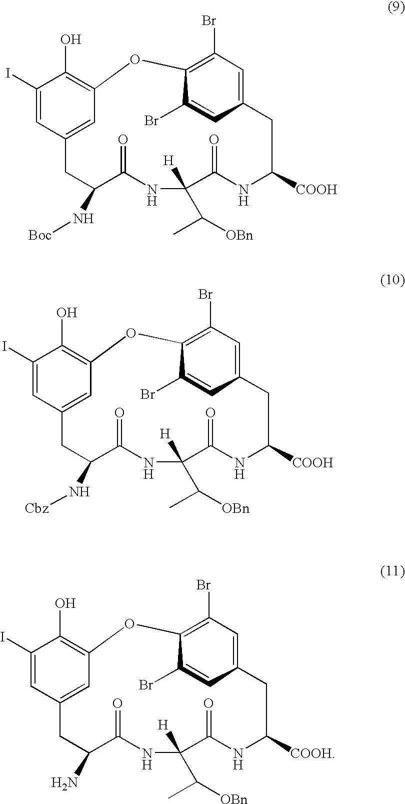

59. The method according to of claim 54, wherein the activity is antibacterial activity.

60. The method according to claim 59, wherein the antibacterial activity is antibacterial activity against methicillin-resistant Staphylococcus aureus (MRSA).

61. A method of inhibiting the growth of bacteria, comprising allowing a compound represented by general formula (III) or a pharmacologically acceptable salt thereof, and imipenem to act in combination with each other: 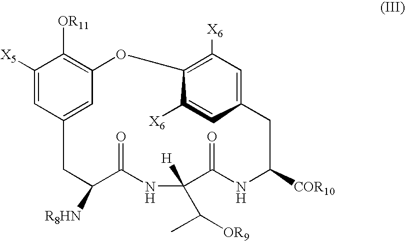
wherein R₈ represents a hydrogen atom, an amido group, a Boc group, a Cbz group, or a linear or branched alkyl group; R₉ represents a hydrogen atom, a benzoyl group, a benzyl group, a modified benzyl group, or a linear or branched alkyl group; R₁₀ represents a hydrogen atom, an —OR₁₂ group, wherein R₁₂ represents a linear or branched alkyl group or an aromatic ring, or an amido group; R₁₁ represents a hydrogen atom, or a linear or branched alkyl group; X₅ and X₆ each represent a halogen atom, wherein X₅ and X₆ may be the same as or different from each other; but when R₉ is a hydrogen atom, R₈ is a hydrogen atom or a Boc group.

62. A method of inhibiting the growth of bacteria, comprising allowing a compound represented by any one of formulae (1) to (5) or a pharmacologically acceptable salt thereof, and imipenem to act in combination with each other:

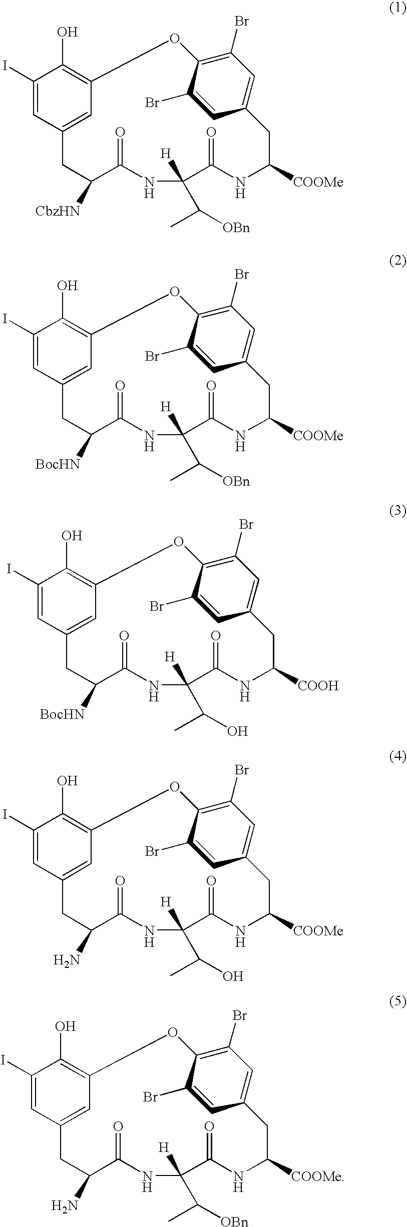

63. A method of inhibiting the growth of bacteria, comprising allowing a compound represented by formula (12) or a pharmacologically acceptable salt thereof, and imipenem to act in combination with each other:

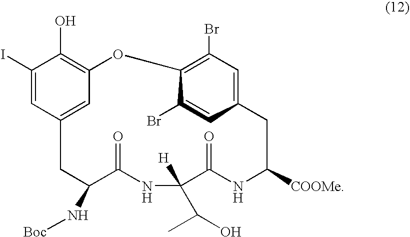

64. A method of inhibiting the growth of bacteria, comprising allowing a compound represented by general formula (II) or a pharmacologically acceptable salt thereof, and imipenem to act in combination with each other: 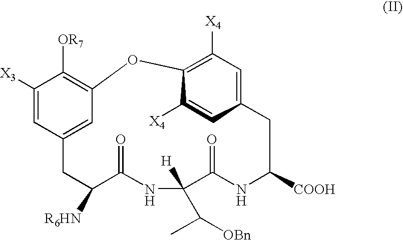
wherein R₆ represents a hydrogen atom, a Boc group, or a Cbz group; R₇ represents a hydrogen atom, or a linear or branched alkyl group; and X₃ and X₄ each represent a halogen atom, wherein X₃ and X₄ may be the same as or different from each other.

65. A method of inhibiting the growth of bacteria comprising allowing a compound represented by any one of formulae (9) to (11) or a pharmacologically acceptable salt thereof, and imipenem to act in combination with each other:

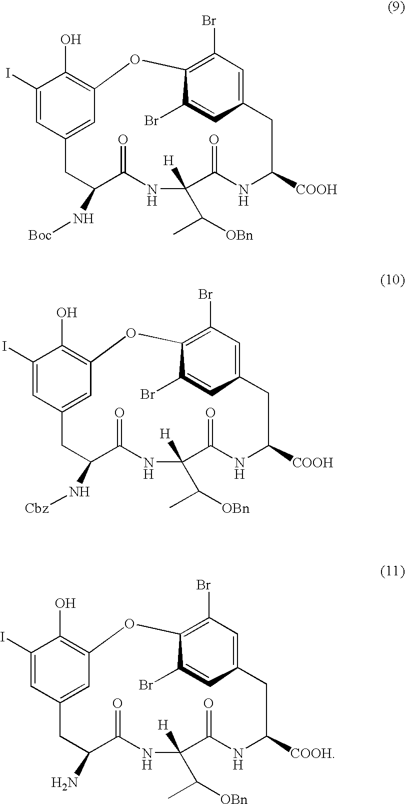

66. The method of inhibiting the growth of bacteria according to claim 61, wherein the bacteria is methicillin-resistant Staphylococcus aureus (MRSA).

67. A method of suppressing the production of cholesterol, comprising administering a compound represented by general formula (IV) or a pharmacologically acceptable salt thereof to a macrophage: 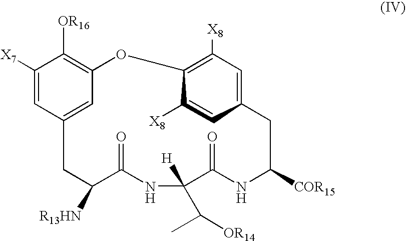
wherein R₁₃ represents an amido group, a Boc group, a Cbz group, an Fmoc group, a BocNH(CH₂)₅CO— group, a BocNHCH(CH₂C₆H₅)CO— group, or a linear or branched alkyl group; R₁₄ represents a benzoyl group, a benzyl group, a modified benzyl group, or a linear or branched alkyl group; R₁₅ represents an —OR₁₇ group, wherein R₁₇ represents a linear or branched alkyl group or an aromatic ring or an amido group; R₁₆ represents a hydrogen atom, or a linear or branched alkyl group; and X₇ and X₈ each represent a halogen atom, wherein X₇ and X₈ may be the same as or different from each other.

68. A method of suppressing the production of cholesterol, comprising administering a compound represented by formula (1) or formula (2), or a pharmacologically acceptable salt thereof to a macrophage:

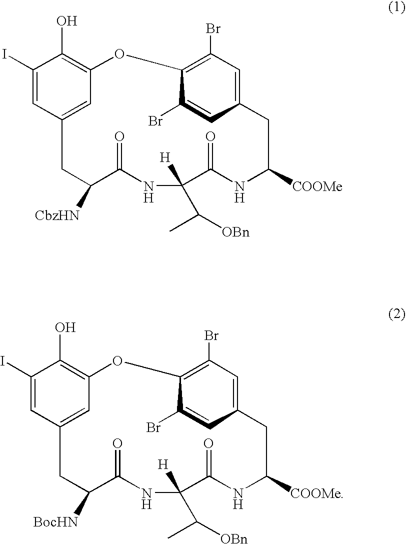

69. A method of suppressing the production of cholesterol, comprising administering a compound represented by any one of formulae (6) to (8) or a pharmacologically acceptable salt thereof to a macrophage:

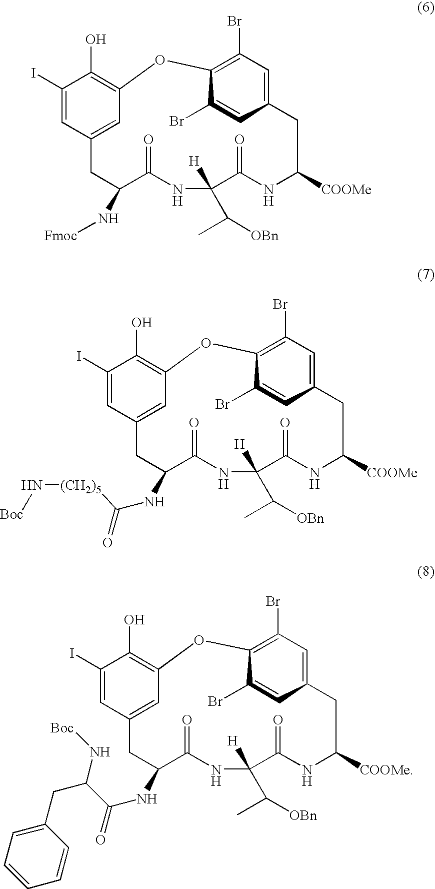

70. A method of inhibiting synthesis of a cholesteryl ester, comprising administering a compound represented by general formula (IV) or a pharmacologically acceptable salt thereof to a macrophage: 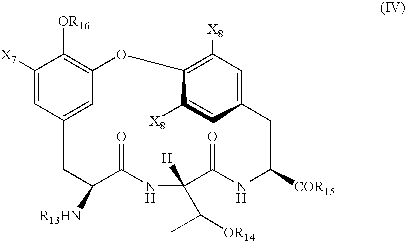
wherein R₁₃ represents an amido group, a Boc group, a Cbz group, an Fmoc group, a BocNH(CH₂)₅CO— group, a BocNHCH(CH₂C₆H₅)CO— group, or a linear or branched alkyl group; R₁₄ represents a benzoyl group, a benzyl group, a modified benzyl group, or a linear or branched alkyl group; R₁₅ represents an —OR₁₇ group, wherein R₁₇ represents a linear or branched alkyl group or an aromatic ring or an amido group; R₁₆ represents a hydrogen atom, or a linear or branched alkyl group; and X₇ and X₈ each represent a halogen atom, wherein X₇ and X₈ may be the same as or different from each other.

71. A method of inhibiting synthesis of a cholesteryl ester, comprising administering a compound represented by formula (1) or formula (2), or a pharmacologically acceptable salt thereof to a macrophage:

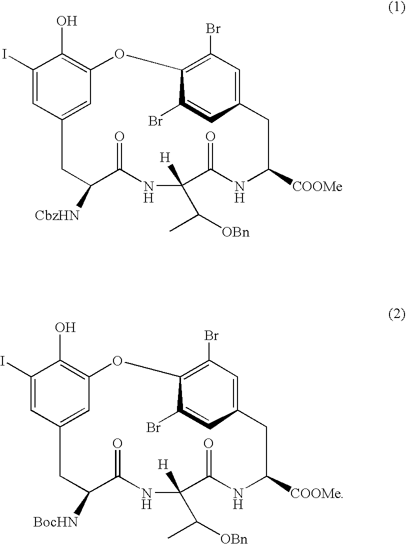

72. A method of inhibiting synthesis of a cholesteryl ester, comprising administering a compound represented by any one of formulae (6) to (8) or a pharmacologically acceptable salt thereof to a macrophage: [chemical structure]
